(19) United States
(12) Patent Application Publication
(10) Pub. No.: US 2026/0078403 A1
(43) Pub. Date: Mar. 19, 2026

(54) ZIKV-BASED GENE DELIVERY SYSTEM

(71) Applicant: The Regents of the University of California, Oakland, CA (US)

(72) Inventors: Alysson Renato Muotri, San Diego, CA (US); Ronaldo Borges, San Diego, CA (US)

(21) Appl. No.: 19/110,023

(22) PCT Filed: Nov. 7, 2023
(86) PCT No.: PCT/US2023/078903

§ 371 Date: Mar. 7, 2025

Related U.S. Application Data
(60) Provisional application No. 63/423,448, filed on Nov. 7, 2022.

(51) Int. Cl. C12N 15/86; A61K 48/00; C07K 14/18; C07K 14/57; C07K 14/705; C07K 16/00; C12N 7/04; C12N 9/12; C12N 15/113

(52) U.S. Cl. CPC C12N 15/86; A61K 48/00; C07K 14/1825; C07K 14/57; C07K 14/705; C07K 16/00; C12N 7/045; C12N 9/1211; C12N 15/113; C12Y 204/02001; C12Y 207/01021; C12N 2310/141; C12N 2770/24143; C12N 2830/002; C12N 2840/203

(57) ABSTRACT

The disclosure provides for recombinantly modified Zika-based vectors that can be used in gene therapy applications.

### CROSS REFERENCE TO RELATED APPLICATIONS

This application claims priority to U.S. Provisional Application Ser. No. 63/423,448, filed Nov. 7, 2022, the disclosures of which are incorporated herein by reference.

### STATEMENT OF GOVERNMENT SUPPORT

This invention was made with Government support under Grant No. NS105969 awarded by the National Institutes of Health. The Government has certain rights in the invention.

### TECHNICAL FIELD

The disclosure provides for recombinantly modified Zika-based vectors that can be used in gene delivery applications.

### INCORPORATION BY REFERENCE OF SEQUENCE LISTING

Accompanying this filing is a Sequence Listing entitled, “00015-418WO1.xml” created on Nov. 7, 2023 and having 60, 793 bytes of data, machine formatted on IBM-PC, MS-Windows operating system. The sequence listing is hereby incorporated by reference in its entirety for all purposes.

### BACKGROUND

The useful application of gene therapy strategies is frequently thwarted by inadequate systems for the delivery and expression of therapeutic transgene within target cell populations. In theory, the concept of gene therapy is simple: delivery of a “good gene” that will ultimately correct the defects of a disease. However, the execution of gene therapy is everything but simple. Indeed, the main problem of gene therapy is the lack of efficient, specific, and safe DNA delivery systems. This is particularly important in brain diseases, such as brain tumors or neurological syndromes caused by mutations in single genes.

### SUMMARY

Provided herein is the development of recombinantly modified ZIKV particles as a gene delivery system. Further provided herein is the use of use of recombinantly modified ZIKV particles disclosed herein for gene therapy applications. The recombinantly modified ZIKV particles of the disclosure can be assembled in vitro using a trans-complementation method with the expression of virus C-prM/M-E-NS1 DNA sequence. The recombinantly modified ZIKV particles of the disclosure is many advantages over existing viral vectors (e.g., Lentiviral vectors, AAV vectors, etc.) including, but not limited to, the ability to cross the blood-brain barrier, cross the mammalian placenta, long-term/persistence in the CNV and CNS, and easy to manipulate. It is expected that the recombinantly modified ZIKV particles disclosed herein are ideally suited as a gene therapy delivery system. More specifically, as a gene therapy delivery system for the brain.

The disclosure provides a recombinantly modified Zika virus (ZIKV)-based particle or vector, comprising an RNA genome having from 5′ to 3′: a 5′ UTR from a Flavivirus; one or more heterologous genes comprising a transgene cassette inserted into or replacing coding sequences for genes selected from the group consisting of a capsid gene (C gene), a pRM/M gene, an envelope gene (E gene), an NS1 gene, and any combination thereof; one or more genes for nonstructural proteins NS1, NS2A, NS2B, NS3, NS4A, NS4B, and/or NS5 from ZIKV; and a 3′ UTR from a Flavivirus. In one embodiment, the ZIKV-based particle or vector does not comprise the genes for protein C, prM/M, and/or protein E from ZIKV. In another or further embodiment, the 3′ UTR is not polyadenylated and is terminated with CU_{OH}. In still another or further embodiment, the 5′ UTR and/or 3′ UTR is from ZIKV. In another or further embodiment, the ZIKV-based particle or vector comprises genes for NS2A, NS2B, NS3, NS4A, NS4B, and NS5 from ZIKV. In another or further embodiment, the ZIKV based particle or vector comprises a capsid; an RNA genome, the RNA genome comprising from 5′ to 3′: a 5′ cap; a 5′ UTR; a transgene cassette replacing nucleotides 61 to 312 of the capsid gene; a prM/M coding sequence; an E protein coding sequence; non-structural protein-coding sequences of NS1, NS2A, NS2B, NS3, NS4A, NS4B and NS5; a 3′ UTR; wherein the transgene cassette is operably linked to a 5′UTR or comprises an internal ribosome binding site; and cis-acting sequences necessary for packaging and replication in a target cell. In another embodiment, the ZIKV-based particle or vector comprises a capsid; an RNA genome, the RNA genome comprising from 5′ to 3′: a 5′ cap; a 5′ UTR; a capsid coding sequence; a transgene cassette replacing nucleotides 16 to 486 of the prM/M gene; an E protein coding sequence; non-structural protein coding sequences of NS1, NS2A, NS2B, NS3, NS4A, NS4B and NS5; a 3′ UTR; wherein the transgene cassette is operably linked to a 5′UTR or comprises an internal ribosome binding site; and cis-acting sequences necessary for packaging and replication in a target cell. In another embodiment, the ZIKV-based particle or vector comprises a capsid; an RNA genome, the RNA genome comprising from 5′ to 3′: a 5′ cap; a 5′ UTR; a capsid coding sequence; a prM/M coding sequence; a transgene cassette replacing nucleotides 16 to 1494 of the Envelope gene; non-structural protein coding sequences of NS1, NS2A, NS2B, NS3, NS4A, NS4B and NS5; a 3′ UTR; wherein the transgene cassette is operably linked to a 5′UTR or comprises an internal ribosome binding site; and cis-acting sequences necessary for packaging and replication in a target cell. In still another embodiment, the ZIKV-based particle or vector comprises a capsid; an RNA genome, the RNA genome comprising from 5′ to 3′: a 5′ cap; a 5′ UTR; a capsid coding sequence; a prM/M coding sequence; an E protein coding sequence; a transgene cassette replacing nucleotides 16 to 894 of the NS1 gene; non-structural protein coding sequences of NS2A, NS2B, NS3, NS4A, NS4B and NS5; a 3′ UTR; wherein the transgene cassette is operably linked to a 5′UTR or comprises an internal ribosome binding site; and cis-acting sequences necessary for packaging and replication in a target cell. In still another embodiment, the ZIKV-based particle or vector comprises: a capsid; an RNA genome, the RNA genome comprising from 5′ to 3′: a 5′ cap; a 5′ UTR; a transgene cassette replacing nucleotides beginning at nucleotide 61 of the capsid gene to nucleotide 894 of the NSA gene; non-structural protein-coding sequences of NS2A, NS2B, NS3, NS4A, NS4B and NS5; a 3′ UTR; wherein the transgene cassette is operably linked to a 5′UTR or comprises an internal ribosome binding site and cis-acting sequences necessary for packaging and replication in a target cell. In another or further embodiment of any of the foregoing, the one or more heterologous genes are used (1) to prevent, treat, or ameliorate a genetic disorder; (2) to replace mutated genes; (3) to fix mutated genes; and/or (4) to make diseased cells more evident to the immune system. In another embodiment, the genetic disorder is a neurodegenerative genetic disorder. In a further embodiment, the neurodegenerative genetic disorder is selected from Alzheimer's disease, amyotrophic lateral sclerosis (ALS), prion disease, Parkinson's disease, Leber congenital amaurosis (LCA), choroideremia, Leber hereditary optic neuropathy (LHON), and Huntington's disease. In still another embodiment, the ZIKV-based particle or vector comprises more than one heterologous gene in the transgene cassette, wherein each heterologous gene is operably linked to a regulatory element. In yet another embodiment, each heterologous gene is operably linked to the same regulatory element. In still another embodiment, each heterologous gene or transgene is operably linked to different regulatory elements. In yet another embodiment, each heterologous gene is separated by a P2A self-cleavable linker coding domain. In another embodiment of any of the foregoing embodiments, the RNA genome is engineered from a polynucleotide having a sequence that is at 988 identical to SEQ ID NO: 1, wherein T is U. In another embodiment of any of the foregoing embodiments, the transgene cassette is from 30 to 4000 bp. In still another embodiment, the regulatory element comprises an internal ribosome entry site (IRES). In another or further embodiment of any of the foregoing, the 5′UTR comprises a sequence that is at least 98% identical to SEQ ID NO:2, wherein T is U. In another or further embodiment of any of the foregoing, the capsid gene comprises a sequence that is at least 98% identical to SEQ ID NO: 3, wherein T is U. In another or further embodiment of any of the foregoing, the prM/M gene comprises a sequence that is at least 98% identical to SEQ ID NO: 4, wherein T is U. In another or further embodiment of any of the foregoing, the envelop gene comprises a sequence that is at least 98% identical to SEQ ID NO:5, wherein T is U. In another or further embodiment of any of the foregoing, the NS1 gene comprises a sequence that is at least 988 identical to SEQ ID NO: 6, wherein T is U. In another or further embodiment of any of the foregoing, NS2A gene comprises a sequence that is at least 98% identical to SEQ ID NO: 7, wherein T is U. In another or further embodiment of any of the foregoing, the NS2B gene comprises a sequence that is at least 98% identical to SEQ ID NO: 8, wherein T is U. In another or further embodiment of any of the foregoing, the NS3 gene comprises a sequence that is at least 98% identical to SEQ ID NO: 9, wherein T is U. In another or further embodiment of any of the foregoing, the NS4A gene comprises a sequence that is at least 988 identical to SEQ ID NO: 10, wherein T is U. In another or further embodiment of any of the foregoing, the NS4B gene comprises a sequence that is at least 98% identical to SEQ ID NO:11, wherein T is U. In another or further embodiment of any of the foregoing, the NS5 gene comprises a sequence that is at least 988 identical to SEQ ID NO: 12, wherein T is U. In another or further embodiment of any of the foregoing, the 3′UTR comprises a sequence that is at least 98% identical to SEQ ID NO: 13, wherein T is U. In another embodiment, heterologous gene encodes a biological response modifier or an immunopotentiating cytokine. In a further embodiment, the immunopotentiating cytokine is selected from the group consisting of interleukins 1 through 38, interferon, tumor necrosis factor (TNF), and granulocyte-macrophage-colony stimulating factor (GM-CSF). In another embodiment, the immunopotentiating cytokine is interferon gamma. In still another embodiment, the heterologous gene encodes a polypeptide that converts a nontoxic prodrug into a toxic drug. In a further embodiment, the polypeptide that converts a nontoxic prodrug into a toxic drug is thymidine kinase, purine nucleoside phosphorylase (PNP), or cytosine deaminase. In another embodiment, the heterologous gene encodes a receptor domain, an antibody, or an antibody fragment. In another embodiment, the heterologous gene comprises an inhibitory polynucleotide. In a further embodiment, the inhibitory polynucleotide comprises a miRNA, RNAi or siRNA sequence. The disclosure also provides a recombinant polynucleotide for producing the recombinant modified ZIKV-based particle or vector of any of the foregoing embodiments. The disclosure also provides a pharmaceutical composition comprising the ZIKA-based particle or vector of the disclosure in a pharmaceutically acceptable carrier. The disclosure also provides a method of delivering a gene or polynucleotide to a cell comprising contacting the cell with the recombinant modified ZIKV-based particle or vector of any of the foregoing embodiments under conditions such that the heterologous polynucleotide is expressed.

The details of one or more embodiments of the disclosure are set forth in the accompanying drawings and the description below. Other features, objects, and advantages will be apparent from the description and drawings, and from the claims.

### BRIEF DESCRIPTION OF DRAWINGS

FIG. 1 presents schematic diagrams of the ZIKV virus genome designed to produce recombinantly modified ZIKV particles carrying reporter genes for efficient delivery by using a CPER strategy.

FIG. 2 provides a plasmid map of pcDNA3.1 (+)-CprMENS1 which can be used in a trans-complementation method which allows for the non-infectious recombinantly modified ZIKV particles to be assembled in vitro.

FIG. 3 provides a schematic overview of the steps required to produce the recombinantly modified ZIKV particles carrying reporter genes by CPER strategy.

FIG. 4 demonstrates the delivery of the mCherry (red) reporter gene, which replaced prM gene, using the nonreplicative ZIKV-derived vector in VERO E6 cells.

FIG. 5 provides a schematic of the generation of the UTR linkers.

### DETAILED DESCRIPTION

As used herein and in the appended claims, the singular forms “a,” “an,” and “the” include plural referents unless the context clearly dictates otherwise. Thus, for example, reference to “a cell” includes a plurality of such cells and reference to “the polynucleotide” includes reference to one or more polynucleotides and so forth.

Also, the use of “or” means “and/or” unless stated otherwise. Similarly, “comprise,” “comprises,” “comprising,” “include,” “includes,” and “including” are interchangeable and not intended to be limiting.

It is to be further understood that where descriptions of various embodiments use the term “comprising,” those skilled in the art would understand that in some specific instances, an embodiment can be alternatively described using language “consisting essentially of” or “consisting of.”

Unless otherwise defined, all technical and scientific terms used herein have the same meaning as commonly understood by one of ordinary skill in the art to which this invention belongs. Allen et al., Remington: The Science and Practice of Pharmacy 22^{nd} ed., Pharmaceutical Press (Sep. 15, 2012); Hornyak et al., Introduction to Nanoscience and Nanotechnology, CRC Press (2008); Singleton and Sainsbury, Dictionary of Microbiology and Molecular Biology 3^{rd} ed., revised ed., J. Wiley & Sons (New York, NY 2006); Smith, March's Advanced Organic Chemistry Reactions, Mechanisms and Structure 7th ed., J. Wiley & Sons (New York, NY 2013); Singleton, Dictionary of DNA and Genome Technology 3^{rd} ed., Wiley-Blackwell (Nov. 28, 2012); and Green and Sambrook, Molecular Cloning: A Laboratory Manual 4^{th} ed., Cold Spring Harbor Laboratory Press (Cold Spring Harbor, NY 2012), provide one skilled in the art with a general guide to many of the terms used in the present application. For references on how to prepare antibodies, see Greenfield, Antibodies A Laboratory Manual 2nd ed., Cold Spring Harbor Press (Cold Spring Harbor NY, 2013); Köhler and Milstein, Derivation of specific antibody-producing tissue culture and tumor lines by cell fusion, Eur. J. Immunol. 1976 Jul. 6 (7): 511-9; Queen and Selick, Humanized immunoglobulins, U.S. Pat. No. 5,585,089 (1996 December); and Riechmann et al., Reshaping human antibodies for therapy, Nature 1988 Mar. 24, 332 (6162): 323-7. All headings and subheading provided herein are solely for ease of reading and should not be construed to limit the invention. Although methods and materials similar or equivalent to those described herein can be used in the practice or testing of the invention, suitable methods and materials are described below. All publications, patent applications, patents, and other references mentioned herein are incorporated by reference in their entirety. In case of conflict, the present specification, including definitions, will control. In addition, the materials, methods, and specific examples are illustrative only and not intended to be limiting.

Any publications disclosed herein and throughout the text are provided solely for their disclosure prior to the filing date of the present application. Nothing herein is to be construed as an admission that the inventors are not entitled to antedate such disclosure by virtue of prior disclosure.

The term “about” when referring to a measurable value such as an amount, a temporal duration, and the like, is meant to encompass variations of ±20% or in some instances ±108, or in some instances ±58, or in some instances ±1%, or in some instances ±0.18 from the specified value, as such variations are appropriate to perform the disclosed methods or describe the compositions herein. Moreover, any value or range (e.g., less than 20 or similar terminology) explicitly includes any integer between such values or up to the value. Thus, for example, “one to five mutations” explicitly includes 1, 2, 3, 4, and/or 5 mutations.

“Cancer” and “cancerous” refer to or describe the physiological condition in mammals that is typically characterized by unregulated cell growth. Examples of cancers include, but are not limited to, B-cell lymphomas (Hodgkin's lymphomas and/or non-Hodgkins lymphomas), T cell lymphomas, myeloma, myelodysplastic syndrome, skin cancer, brain tumor, breast cancer, colon cancer, rectal cancer, esophageal cancer, anal cancer, cancer of unknown primary site, endocrine cancer, testicular cancer, lung cancer, hepatocellular cancer, gastric cancer, pancreatic cancer, cervical cancer, ovarian cancer, liver cancer, bladder cancer, cancer of the urinary tract, cancer of reproductive organs thyroid cancer, renal cancer, carcinoma, melanoma, head and neck cancer, brain cancer (e.g., glioblastoma multiforme), prostate cancer, including but not limited to androgen-dependent prostate cancer and androgen-independent prostate cancer, and leukemia. Other cancer and cell proliferative disorders will be readily recognized in the art. The terms “tumor” and “cancer” are used interchangeably herein, e.g., both terms encompass solid and liquid, e.g., diffuse or circulating, tumors. As used herein, the term “cancer” or “tumor” includes premalignant, as well as malignant cancers and tumors.

Typically a recombinant ZIKA vector of the disclosure is modified to include a “cassette” or “transgene cassette”, which typically contains at least one heterologous gene or polynucleotide to be expressed. In some embodiments, the heterologous gene or polynucleotide is operably linked to elements that allow effective expression (e.g., a promoter, IRES, or a read-through element that allows transcription and/or translation of the heterologous sequence).

The term DNA “control sequences” refers collectively to promoter sequences, polyadenylation signals, transcription termination sequences, upstream regulatory domains, origins of replication, internal ribosome entry sites (“IRES”), initiators of translation, enhancers, and the like, which collectively provide for the replication, transcription and/or translation of a coding sequence in a recipient cell. Not all of these control sequences need always be present so long as the selected coding sequence is capable of being replicated, transcribed and/or translated in an appropriate host cell.

The term “heterologous,” as it relates to nucleic acid sequences such as coding sequences and control sequences, denotes sequences that are not normally joined together, and/or are not normally associated with a particular cell or genetic entity. Thus, a “heterologous” region of a nucleic acid construct or a vector is a segment of nucleic acid within or attached to another nucleic acid molecule that is not found in association with the other molecule in nature. For example, a heterologous region of a nucleic acid construct could include a coding sequence flanked by sequences not found in association with the coding sequence in nature. Another example of a heterologous coding sequence is a construct where the coding sequence itself is not found in nature (e.g., synthetic sequences having codons different from the native gene). Similarly, a cell transformed with a construct which is not normally present in the cell would be considered heterologous for purposes of this invention. Allelic variation or naturally occurring mutational events do not give rise to heterologous DNA, as used herein.

A heterologous gene or polynucleotide contained in a cassette can comprise any number of sequences to be expressed. In some embodiments, the heterologous polynucleotide or gene is a therapeutic gene or polynucleotide. In some embodiments, the therapeutic polynucleotide encodes a therapeutic polypeptide. In other embodiments, the heterologous polynucleotide can encode a diagnostic or marker polypeptide. In some embodiments, the diagnostic or marker polypeptide encodes a fluorescent, luminescent or enzymatic polypeptide. A heterologous polynucleotide or gene can be selected from the group consisting of a therapeutic polynucleotide encoding a polypeptide (e.g., an antibody, antibody fragment, diabody, prodrug activator such as a thymidine kinase or cytosine deaminase), a therapeutic nucleic acid (e.g., a siRNA, miRNA, antisense oligonucleotide (ASO) and the like), a polynucleotide that encodes a defective gene to be replaced or complemented, a growth factor, a chimeric antigen receptor (including 1^{st}, 2^{nd} and later generations of CARs). In yet another or further embodiment, the heterologous polynucleotide can comprise a cytokine such as an interleukin, interferon-gamma, or the like. Cytokines that may expressed from a retroviral vector of the disclosure include but are not limited to, IL-1alpha, IL-1beta, IL-2, IL-3, IL-4, IL-5, IL-6, IL-7, IL-8, IL-9, IL-10, IL-11, IL-12, IL-13, IL-14, IL-15, IL-16, IL-17, IL-18, IL-19, IL-20, IL-21, IL-22, IL-23, IL-24, IL-25, IL-26, IL-27, IL-28, IL-29, IL-30, IL-31, IL-32, IL-33, IL-34, IL-35, IL-36, IL-37, IL-38, anti-CD40, CD40L, IFN-gamma and TNF-alpha, soluble forms of TNF-alpha, lymphotoxin-alpha (LT-alpha, also known as TNF-beta), LT-beta (found in complex heterotrimer LT-alpha2-beta), OPGL, FasL, CD27L, CD30L, CD40L, 4-1BBL, DCR3, OX40L, TNF-gamma (International Publication No. WO 96/14328), AIM-I (International Publication No. WO 97/33899), endokine-alpha (International Publication No. WO 98/07880), OPG, and neutrokine-alpha (International Publication No. WO 98/18921, OX40, and nerve growth factor (NGF), and soluble forms of Fas, CD30, CD27, CD40 and 4-IBB, TR2 (International Publication No. WO 96/34095), DR3 (International Publication No. WO 97/33904), DR4 (International Publication No. WO 98/32856), TR5 (International Publication No. WO 98/30693), TRANK, TR9 (International Publication No. WO 98/56892), TR10 (International Publication No. WO 98/54202), 312C2 (International Publication No. WO 98/06842), and TR12, and soluble forms CD154, CD70, and CD153. Angiogenic proteins may be useful in some embodiments, particularly for protein production from cell lines. Such angiogenic factors include, but are not limited to, Glioma Derived Growth Factor (GDGF), Platelet Derived Growth Factor-A (PDGF-A), Platelet Derived Growth Factor-B (PDGF-B), Placental Growth Factor (PIGF), Placental Growth Factor-2 (PIGF-2), Vascular Endothelial Growth Factor (VEGF), Vascular Endothelial Growth Factor-A (VEGF-A), Vascular Endothelial Growth Factor-2 (VEGF-2), Vascular Endothelial Growth Factor B (VEGF-3), Vascular Endothelial Growth Factor B-1 86 (VEGF-B186), Vascular Endothelial Growth Factor-D (VEGF-D), Vascular Endothelial Growth Factor-D (VEGF-D), and Vascular Endothelial Growth Factor-E (VEGF-E). Fibroblast Growth Factors may be delivered by a vector of the disclosure and include, but are not limited to, FGF-1, FGF-2, FGF-3, FGF-4, FGF-5, FGF-6, FGF-7, FGF-8, FGF-9, FGF-10, FGF-11, FGF-12, FGF-13, FGF-14, and FGF-15. Hematopoietic growth factors may be delivered using vectors of the disclosure, such growth factors include, but are not limited to, granulocyte-macrophage colony-stimulating factor (GM-CSF) (sargramostim), granulocyte colony-stimulating factor (G-CSF) (filgrastim), macrophage colony-stimulating factor (M-CSF, CSF-1) erythropoietin (epoetin alfa), stem cell factor (SCF, c-kit ligand, steel factor), megakaryocyte colony-stimulating factor, PIXY321 (a GMCSF/IL-3) fusion protein and the like.

Where the heterologous polynucleotide encodes a cancer therapeutic, the vector can be used alone or in combination with first-line chemotherapeutic agents.

Transgenes (e.g., the heterologous sequence to be expressed) can be inserted into a recombinant viral genome in a number of locations, including into one or more ZIKA virus genome domains selected from the C-gene, prm/M gene, E-gene, and/or the NS1 gene or any combination of the adjacent gene of the foregoing. Internal IRES sequences, 2A self-cleaving peptide coding sequences (where more than one heterologous polynucleotide is present) or other regulatory elements to promote translation and/or transcription of the heterologous polynucleotide(s) may be present and operably linked to the heterologous polynucleotide(s).

A “coding sequence” or a sequence which “encodes” a selected polypeptide, is a nucleic acid molecule which is transcribed (in the case of DNA) and translated (in the case of mRNA) into a polypeptide in vivo when placed under the control of appropriate regulatory sequences. The boundaries of the coding sequence are determined by a start codon at the 5′ (amino) terminus and a translation stop codon at the 3′ (carboxy) terminus. A transcription termination sequence may be located 3′ to the coding sequence.

“Chemotherapeutic agents” are compounds that are known to be of use in chemotherapy for cancer. Non-limiting examples of chemotherapeutic agents can include alkylating agents such as thiotepa and CYTOXAN® cyclosphosphamide; alkyl sulfonates such as busulfan, improsulfan and piposulfan; aziridines such as benzodopa, carboquone, meturedopa, and uredopa; ethylenimines and methylamelamines including altretamine, triethylenemelamine, trietylenephosphoramide, triethiylenethiophosphoramide and trimethylolomelamine; acetogenins (especially bullatacin and bullatacinone); a camptothecin (including the synthetic analogue topotecan); bryostatin; callystatin; CC-1065 (including its adozelesin, carzelesin and bizelesin synthetic analogues); cryptophycins (particularly cryptophycin 1 and cryptophycin 8); dolastatin; duocarmycin (including the synthetic analogues, KW-2189 and CB1-TM1); eleutherobin; pancratistatin; a sarcodictyin; spongistatin; nitrogen mustards such as chlorambucil, chlornaphazine, cholophosphamide, estramustine, ifosfamide, mechlorethamine, mechlorethamine oxide hydrochloride, melphalan, novembichin, phenesterine, prednimustine, trofosfamide, uracil mustard; nitrosureas such as carmustine, chlorozotocin, fotemustine, lomustine, nimustine, and ranimnustine; antibiotics such as the enediyne antibiotics (e.g., calicheamicin, especially calicheamicin gammalI and calicheamicin omegaIl (see, e.g., Agnew, Chem. Intl. Ed. Engl., 33:183-186 (1994)); dynemicin, including dynemicin A; bisphosphonates, such as clodronate; an esperamicin; as well as neocarzinostatin chromophore and related chromoprotein enediyne antibiotic chromophores), aclacinomysins, actinomycin, authramycin, azaserine, bleomycins, cactinomycin, carabicin, caminomycin, carzinophilin, chromomycinis, dactinomycin, daunorubicin, detorubicin, 6-diazo-5-oxo-L-norleucine, ADRIAMYCIN® doxorubicin (including morpholino-doxorubicin, cyanomorpholino-doxorubicin, 2-pyrrolino-doxorubicin and deoxydoxorubicin), epirubicin, esorubicin, idarubicin, marcellomycin, mitomycins such as mitomycin C, mycophenolic acid, nogalamycin, olivomycins, peplomycin, potfiromycin, puromycin, quelamycin, rodorubicin, streptonigrin, streptozocin, tubercidin, ubenimex, zinostatin, zorubicin; anti-metabolites such as methotrexate and 5-fluorouracil (5-FU); folic acid analogues such as denopterin, methotrexate, pteropterin, trimetrexate; purine analogs such as fludarabine, 6-mercaptopurine, thiamiprine, thioguanine; pyrimidine analogs such as ancitabine, azacitidine, 6-azauridine, carmofur, cytarabine, dideoxyuridine, doxifluridine, enocitabine, floxuridine; androgens such as calusterone, dromostanolone propionate, epitiostanol, mepitiostane, testolactone; anti-adrenals such as aminoglutethimide, mitotane, trilostane; folic acid replenisher such as frolinic acid; aceglatone; aldophosphamide glycoside; aminolevulinic acid; eniluracil; amsacrine; bestrabucil; bisantrene; edatraxate; defofamine; demecolcine; diaziquone; elformithine; elliptinium acetate; an epothilone; etoglucid; gallium nitrate; hydroxyurea; lentinan; lonidainine; maytansinoids such as maytansine and ansamitocins; mitoguazone; mitoxantrone; mopidanmol; nitraerine; pentostatin; phenamet; pirarubicin; losoxantrone; podophyllinic acid; 2-ethylhydrazide; procarbazine; PSK® polysaccharide complex (JHS Natural Products, Eugene, Oreg.); razoxane; rhizoxin; sizofuran; spirogermanium; tenuazonic acid; triaziquone; 2,2′,2″-trichlorotriethylamine; trichothecenes (especially T-2 toxin, verracurin A, roridin A and anguidine); urethan; vindesine; dacarbazine; mannomustine; mitobronitol; mitolactol; pipobroman; gacytosine; arabinoside (“Ara-C”); cyclophosphamide; thiotepa; taxoids, e.g., TAXOL® paclitaxel (Bristol-Myers Squibb Oncology, Princeton, N. J.), ABRAXANE® Cremophor-free, albumin-engineered nanoparticle formulation of paclitaxel (American Pharmaceutical Partners, Schaumberg, Ill.), and TAXOTERE® doxetaxel (Rhone-Poulenc Rorer, Antony, France); chloranbucil; GEMZAR® gemcitabine; 6-thioguanine; mercaptopurine; methotrexate; platinum analogs such as cisplatin, oxaliplatin and carboplatin; vinblastine; platinum; etoposide (VP-16); ifosfamide; mitoxantrone; vincristine; NAVELBINE; vinorelbine; novantrone; teniposide; edatrexate; daunomycin; aminopterin; xeloda; ibandronate; irinotecan (Camptosar, CPT-11) (including the treatment regimen of irinotecan with 5-FU and leucovorin); topoisomerase inhibitor RFS 2000; difluoromethylornithine (DMFO); retinoids such as retinoic acid; capecitabine; combretastatin; leucovorin (LV); oxaliplatin, including the oxaliplatin treatment regimen (FOLFOX); lapatinib (Tykerb); inhibitors of PKC-alpha, Raf, H-Ras, EGFR (e.g., erlotinib (Tarceva®)) and VEGF-A that reduce cell proliferation and pharmaceutically acceptable salts, acids or derivatives of any of the above or combinations thereof.

The terms “express” and “expression” mean allowing or causing the information in a gene or DNA sequence to become manifest, for example, producing a protein by activating the cellular functions involved in the transcription and translation of a corresponding gene or DNA sequence or in the case of inhibitor RNA (RNAi) transcribing the RNAi molecule such that is processed and capable of inhibiting expression of a target gene.

A DNA sequence is expressed in or by a cell to form an “expression product” such as a protein. The expression product itself, e.g. the resulting protein, may also be said to be “expressed” by the cell. A polynucleotide or polypeptide is expressed recombinantly, for example, when it is expressed or produced in a foreign host cell under the control of a foreign or native promoter, or in a native host cell under the control of a foreign promoter. In instances where an RNA sequence is delivered, the RNA sequence is expressed by translating the RNA to produce an encoded product such as a polypeptide or biologically active nucleic acid moiety (e.g., siRNA, etc.).

As mentioned above, in some instances the term “express” includes the production of inhibitory RNA molecules (RNAi). The expression of such molecules do not involve the translation machinery of the cell but rather utilize machinery in a cell to modify a host cell's gene expression. In some embodiments, a recombinant viral vector of the disclosure can be modified to express a coding sequence (e.g., a protein), express an RNAi molecule, or express both a coding sequence (e.g., express a protein) and express and RNAi molecule.

“Genetically modified cells”, “redirected cells”, “genetically engineered cells,” or “modified cells” as used herein refer to cells that have been modified to express a transgene or which contain a foreign polynucleotide that expresses a heterologous molecule not naturally found in the cell or cells.

The terms “genome particles (gp),” or “genome equivalents,” as used in reference to a viral titer, refer to the number of virions containing the recombinantly modified ZIKA genome, regardless of infectivity or functionality. The number of genome particles in a particular vector preparation can be measured by procedures such as those described in the Examples herein, or for example, in Clark et al., Hum. Gene Ther. (1999) 10:1031-1039; and Veldwijk et al., Mol. Ther. (2002) 6:272-278.

An internal ribosome entry sites (“IRES”) refers to a segment of nucleic acid that promotes the entry or retention of a ribosome during the translation of a coding sequence, usually 3′ to the IRES. In some embodiments, the IRES may comprise a splice acceptor/donor site, however, preferred IRESs lack a splice acceptor/donor site. Normally, the entry of ribosomes into messenger RNA takes place via the cap located at the 5′ end of all eukaryotic mRNAs (including the 5′UTR of the ZIKAV). However, there are exceptions to this universal rule. The absence of a cap in some viral mRNAs suggests the existence of alternative structures permitting the entry of ribosomes at an internal site of these RNAs. To date, a number of these structures, designated IRES on account of their function, have been identified in the 5′ noncoding region of uncapped viral mRNAs, such as that of picornaviruses, in particular the poliomyelitis virus (Pelletier et al., 1988, Mol. Cell. Biol., 8, 1103-1112) and the EMCV virus (encephalo-myocarditis virus (Jang et al., J. Virol., 62, 2636-2643 1988; B. T. Baranick et al., Proc Natl Acad Sci USA. 105:4733-8, 2008). The disclosure provides the use of an IRES in the context of a replication-competent retroviral vector.

By “isolated”, when referring to a nucleotide sequence, is meant that the indicated molecule is present in the substantial absence of other biological macromolecules of the same type. Thus, an “isolated nucleic acid molecule which encodes a particular polypeptide” refers to a nucleic acid molecule that is substantially free of other nucleic acid molecules that do not encode the subject polypeptide; however, the molecule may include some additional bases or moieties that do not deleteriously affect the basic characteristics of the composition.

“Mammal” as used herein refers to any member of the class Mammalia, including, without limitation, humans and nonhuman primates such as chimpanzees and other apes and monkey species; farm animals such as cattle, sheep, pigs, goats, and horses; domestic mammals such as dogs and cats; laboratory animals including rodents such as mice, rats and guinea pigs, and the like. The term does not denote a particular age or sex. Thus, adult and newborn subjects, as well as fetuses, whether male or female, are intended to be included within the scope of this term.

The term “operably linked” refers to functional linkage or association between a first component and a second component such that each component can be functional. For example, operably linked includes the association between a regulatory sequence and a heterologous nucleic acid sequence, resulting in the expression of the latter. For example, a first nucleic acid sequence is operably linked with a second nucleic acid sequence when the first nucleic acid sequence is placed in a functional relationship with the second nucleic acid sequence. In the context of two polypeptides that are operably linked, the first polypeptide functions in the manner it would be independent of any linkage, and the second polypeptide functions as it would absent a linkage between the two.

A “2A peptide or 2A peptide-like sequence” refers to a peptide having the consensus sequence of SEQ ID NO: 14, a sequence that contains the consensus sequence of SEQ ID NO: 14. A sequence that “encodes” a 2A peptide or 2A peptide-like sequence is a polynucleotide sequence that encodes a 2A peptide or peptide-like sequence having, e.g., the consensus sequence of SEQ ID NO: 14. The coding sequence is operably linked to and placed, in one embodiment, between two heterologous sequences, such that once the sequence is translated that two polypeptides are self-cleaved from one another to produce a first polypeptide and a second distinct polypeptide.

Several viruses, including picornaviruses and encephalomyocarditis virus, encode 2A or 2A-like peptides in their genomes to mediate multiple protein expressions from a single ORF. 2A peptides are typically about 16-18 amino acids in sequence and share the consensus motif (D[V/I] EXNPGP (SEQ ID NO: 14), wherein X is any amino acid). When the 2A peptide is encoded between ORFs in an artificial multicistronic mRNA, it causes the ribosome to halt at the C-terminus of 2A peptide in the translating polypeptide, thus resulting in the separation of polypeptides derived from each ORF (Doronina et al., 2008). The separation point is at the C-terminus of 2A, with the first amino acid of the downstream ORF being proline. The unique features of 2A peptide have led to its utilization as a molecular tool for multiple-protein expression from a single multicistronic mRNA configuration. 2A peptides have near 100% “separation” efficiency in their native contexts and often have lower “separation” efficiencies when they are introduced into non-native sequences. Other 2A-like sequences found in different classes of viruses have also been shown to achieve ˜85% “separation” efficiency in non-native sequences (Donnelly et al., 1997). There are many 2A-like sequences that can be used in the methods and composition of this disclosure for expressing transgenes.

The phrase “non-dividing” cell refers to a cell that does not go through mitosis. Non-dividing cells may be blocked at any point in the cell cycle, (e.g., G₀/G₁, G_{1/s}, G_{2/M}), so long as the cell is not actively dividing. For ex vivo infection, a dividing cell can be treated to block cell division by standard techniques used by those of skill in the art, including, irradiation, aphidocolin treatment, serum starvation, and contact inhibition. However, it should be understood that ex vivo infection is often performed without blocking the cells since many cells are already arrested (e.g., stem cells). Examples of pre-existing non-dividing cells in the body include neuronal, muscle, liver, skin, heart, lung, and bone marrow cells, and their derivatives. For dividing cells, oncoretroviral vectors can be used.

By “dividing” cell is meant a cell that undergoes active mitosis, or meiosis. Such dividing cells include stem cells, skin cells (e.g., fibroblasts and keratinocytes), gametes, and other dividing cells known in the art. Of particular interest and encompassed by the term dividing cell are cells having cell proliferative disorders, such as neoplastic cells. The term “cell proliferative disorder” refers to a condition characterized by an abnormal number of cell divisions. The condition can include both hypertrophic (the continual multiplication of cells resulting in an overgrowth of a cell population within a tissue) and hypotrophic (a lack or deficiency of cells within a tissue) cell growth or an excessive influx or migration of cells into an area of a body. The cell populations are not necessarily transformed, tumorigenic or malignant cells, but can include normal cells as well. Cell proliferative disorders include disorders associated with an overgrowth of connective tissues, such as various fibrotic conditions, including scleroderma, arthritis and liver cirrhosis. Cell proliferative disorders include neoplastic disorders such as head and neck carcinomas. Head and neck carcinomas would include, for example, carcinoma of the mouth, esophagus, throat, larynx, thyroid gland, tongue, lips, salivary glands, nose, paranasal sinuses, nasopharynx, superior nasal vault and sinus tumors, esthesioneuroblastoma, squamous cell cancer, malignant melanoma, sinonasal undifferentiated carcinoma (SNUC), brain (including glioblastomas such as glioblastoma multiforme) or blood neoplasia. Also included are carcinomas of the regional lymph nodes including cervical lymph nodes, prelaryngeal lymph nodes, pulmonary juxtaesophageal lymph nodes, and submandibular lymph nodes (Harrison's Principles of Internal Medicine (eds., Isselbacher, et al., McGraw-Hill, Inc., 13th Edition, pp 1850-1853, 1994). Other cancer types, include, but are not limited to, lung cancer, colon-rectum cancer, breast cancer, prostate cancer, urinary tract cancer, uterine cancer lymphoma, oral cancer, pancreatic cancer, leukemia, melanoma, stomach cancer, skin cancer and ovarian cancer. The cell proliferative disease also includes rheumatoid arthritis (O'Dell NEJM 350:2591 2004) and other auto-immune disorders (Mackay et al NEJM 345:340 2001) that are often characterized by inappropriate proliferation of cells of the immune system.

“Percent identity” in the context of two or more nucleic acids or polypeptide sequences, refers to two or more sequences that share a degree of similarity. Two sequences are “substantially identical” if two sequences have a specified percentage of amino acid residues or nucleotides that are the same (e.g., 60% identity, optionally 70%, 71%. 72%. 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% identity over a specified region, or, when not specified, over the entire sequence), when compared and aligned for maximum correspondence over a comparison window, or designated region as measured using one of the following sequence comparison algorithms or by manual alignment and visual inspection. Optionally, the identity exists over a region that is at least about 50 nucleotides (or 10 amino acids) in length, or more preferably over a region that is 100 to 500 or 1000 or more nucleotides (or 20, 50, 200, or more amino acids) in length.

For sequence comparison, generally, one sequence acts as a reference sequence, to which test sequences are compared. When using a sequence comparison algorithm, test and reference sequences are entered into a computer, subsequence coordinates are designated, if necessary, and sequence algorithm program parameters are designated. Default program parameters can be used, or alternative parameters can be designated. The sequence comparison algorithm then calculates the percent sequence identities for the test sequences relative to the reference sequence, based on the program parameters. Methods of alignment of sequences for comparison are well known in the art. Optimal alignment of sequences for comparison can be conducted, e.g., by the local homology algorithm of Smith and Waterman, (1970) Adv. Appl. Math. 2: 482c, by the homology alignment algorithm of Needleman and Wunsch, (1970) J. Mol. Biol. 48:443, by the search for similarity method of Pearson and Lipman, (1988) Proc. Nat'l. Acad. Sci. USA 85:2444, by computerized implementations of these algorithms (GAP, BESTFIT, FASTA, and TFASTA in the Wisconsin Genetics Software Package, Genetics Computer Group, 575 Science Dr., Madison, WI), or by manual alignment and visual inspection (see, e.g., Brent et al., (2003) Current Protocols in Molecular Biology).

Two examples of algorithms that can be used for determining percent sequence identity and sequence similarity are the BLAST and BLAST 2.0 algorithms, which are described in Altschul et al., (1977) Nuc. Acids Res. 25:3389-3402; and Altschul et al., (1990) J. Mol. Biol. 215:403-410, respectively. Software for performing BLAST analyses is publicly available through the National Center for Biotechnology Information.

The percent identity between two sequences can also be determined using the algorithm of E. Meyers and W. Miller, (1988) Comput. Appl. Biosci. 4:11-17) which has been incorporated into the ALIGN program (version 2.0), using a PAM120 weight residue table, a gap length penalty of 12 and a gap penalty of 4. In addition, the percent identity between two sequences can be determined using the Needleman and Wunsch (1970) J. Mol. Biol. 48:444-453) algorithm, which has been incorporated into the GAP program in the GCG software package (available at www.gcg.com), using either a Blossom 62 matrix or a PAM250 matrix, and a gap weight of 16, 14, 12, 10, 8, 6, or 4 and a length weight of 1, 2, 3, 4, 5, or 6.

The term “polynucleotide”, “nucleic acid”, or “recombinant nucleic acid” refers to polymers of nucleotides such as deoxyribonucleic acid (DNA), and, where appropriate, ribonucleic acid (RNA). Where a sequence is presented with a “T” (thymine), the same sequence can be converted to RNA by replacing the “T” (thymine) with a “U” (uracil).

A “protein” or “polypeptide”, which terms are used interchangeably herein, comprises one or more chains of chemical building blocks called amino acids that are linked together by chemical bonds called peptide bonds.

A “native” polypeptide, such as a survival motor neuron (SMN) polypeptide, refers to a polypeptide having the same amino acid sequence as the corresponding molecule derived from nature. Such native sequences can be isolated from nature or can be produced by recombinant means. The term “native” sequence specifically encompasses naturally occurring truncated or secreted forms of the specific molecule (e.g., an extracellular domain sequence), naturally occurring variant forms (e.g., alternatively spliced forms), and naturally occurring allelic variants of the polypeptide. In various embodiments of the invention, the native molecules disclosed herein are mature or full-length native sequences comprising the full-length amino acid sequences shown in the accompanying figures. However, while some of the molecules disclosed in the accompanying figures begin with methionine residues designated as amino acid position 1 in the figures, other methionine residues located either upstream or downstream from amino acid position 1 in the figures may be employed as the starting amino acid residue for the particular molecule. Alternatively, depending on the expression system used, the molecules described herein may lack an N-terminal methionine.

The term “promoter” is used herein in its ordinary sense to refer to a nucleotide region comprising a DNA regulatory sequence, wherein the regulatory sequence is derived from a gene that is capable of binding RNA polymerase and initiating transcription of a downstream (3′-direction) coding sequence. Transcription promoters can include “inducible promoters” (where expression of a polynucleotide sequence operably linked to the promoter is induced by an analyte, cofactor, regulatory protein, etc.), “repressible promoters” (where expression of a polynucleotide sequence operably linked to the promoter is induced by an analyte, cofactor, regulatory protein, etc.), and “constitutive promoters.”

As used herein, the term “RNA interference” (RNAi) refers to the process of sequence-specific post-transcriptional gene silencing mediated by short interfering nucleic acids (siRNAs or microRNAs (miRNA)). The term “agent capable of mediating RNA interference” refers to siRNAs as well as DNA and RNA vectors that encode siRNAs when transcribed within a cell. The term siRNA or miRNA is meant to encompass any nucleic acid molecule that is capable of mediating sequence-specific RNA interference, for example short interfering RNA (siRNA), double-stranded RNA (dsRNA), micro-RNA (miRNA), short hairpin RNA (shRNA), short interfering oligonucleotide, short interfering nucleic acid, short interfering modified oligonucleotide, chemically-modified siRNA, post-transcriptional gene silencing RNA (ptgsRNA), and others.

Suitable range for designing stem lengths of a hairpin duplex, includes stem lengths of 20-30 nucleotides, 30-50 nucleotides, 50-100 nucleotides, 100-150 nucleotides, 150-200 nucleotides, 200-300 nucleotides, 300-400 nucleotides, 400-500 nucleotides, 500-600 nucleotides, and 600-700 nucleotides. Suitable range for designing loop lengths of a hairpin duplex includes loop lengths of 4-25 nucleotides, 25-50 nucleotides, or longer if the stem length of the hair duplex is substantial. In certain contexts, hairpin structures with duplexed regions that are longer than 21 nucleotides may promote effective siRNA-directed silencing, regardless of the loop sequence and length.

The terms “subject”, “individual” or “patient” are used interchangeably herein and refer to a vertebrate, preferably a mammal. Mammals include but are not limited to, murines, rodents, simians, humans, farm animals, sport animals, and companion animals (e.g., dogs, cats, rabbits, etc.).

The term “transfection” is used to refer to the uptake of foreign DNA and/or RNA by a cell, and a cell has been “transfected” when exogenous DNA and/or RNA has been introduced inside the cell membrane. A number of transfection techniques are generally known in the art. See, e.g., Graham et al. (1973) Virology, 1:456, Sambrook et al. (1989) Molecular Cloning, a laboratory manual, Cold Spring Harbor Laboratories, New York, Davis et al. (1986) Basic Methods in Molecular Biology, Elsevier, and Chu et al. (1981) Gene 1:197. Such techniques can be used to introduce one or more exogenous DNA moieties into suitable host cells.

The term “therapeutic effect” refers to a biological effect which can be manifested by various means, including but not limited to, e.g., decrease in tumor volume, a decrease in the number of cancer cells, a decrease in the number of metastases, an increase in life expectancy, decrease in cancer cell proliferation, decrease in cancer cell survival, decrease in the titer of the infectious agent, a decrease in colony counts of the infectious agent, amelioration of various physiological symptoms associated with a disease condition. A “therapeutic effect” can also be manifested by the ability of the peptides, polynucleotides, cells, and antibodies to prevent the occurrence of disease in the first place or in the prevention of relapse of the disease. As used herein, the term “ameliorate” is synonymous with “alleviate” and means to reduce or lighten. For example, one may ameliorate the symptoms of a disease or disorder by making the disease or symptoms of the disease less severe.

“Tissue-specific regulatory elements” are regulatory elements (e.g., promoters) that are capable of driving transcription of a gene in one tissue while remaining largely “silent” in other tissue types. It will be understood, however, that tissue-specific promoters may have a detectable amount of “background” or “base” activity in those tissues where they are expected to be silent. The degree to which a promoter is selectively activated in a target tissue can be expressed as a selectivity ratio (activity in a target tissue/activity in a control tissue). In this regard, a tissue-specific promoter useful in the practice of the disclosure typically has a selectivity ratio of greater than about 5. Preferably, the selectivity ratio is greater than about 15.

It will be further understood that certain promoters, while not restricted in activity to a single tissue type, may nevertheless show selectivity in that they may be active in one group of tissues, and less active or silent in another group. Such promoters are also termed “tissue-specific,” and are contemplated for use with the disclosure. For example, promoters that are active in a variety of central nervous system (CNS) neurons may be therapeutically useful in protecting against damage due to stroke, which may affect any of a number of different regions of the brain.

“Treatment” and “treating,” as used herein, refer to both therapeutic treatment and prophylactic or preventative measures, wherein the object is to prevent or slow down (lessen) the targeted pathologic condition, prevent the pathologic condition, pursue or obtain beneficial results, or lower the chances of the individual developing the condition even if the treatment is ultimately unsuccessful. Those in need of treatment include those already with the condition as well as those prone to have the condition or those in whom the condition is to be prevented.

The term “transgene” refers to a polynucleotide that is introduced into a cell and is capable of being transcribed into RNA and/or translated under appropriate conditions. In one aspect, it confers a desired property to a cell into which it was introduced, or otherwise leads to a desired therapeutic or diagnostic outcome.

By “variant” is meant an active polypeptide as defined herein having at least about 80% amino acid sequence identity with the corresponding full-length native sequence, a polypeptide lacking the signal peptide, an extracellular domain of a polypeptide, with or without a signal peptide, or any other fragment of a full-length polypeptide sequence as disclosed herein. Such polypeptide variants include, for instance, polypeptides wherein one or more amino acid residues are added or deleted, at the N- and/or C-terminus of the full-length native amino acid sequence. Ordinarily, a variant will have at least about 80% amino acid sequence identity, alternatively at least about 81% amino acid sequence identity, alternatively at least about 82% amino acid sequence identity, alternatively at least about 83% amino acid sequence identity, alternatively at least about 84% amino acid sequence identity, alternatively at least about 85% amino acid sequence identity, alternatively at least about 86% amino acid sequence identity, alternatively at least about 87% amino acid sequence identity, alternatively at least about 88% amino acid sequence identity, alternatively at least about 89% amino acid sequence identity, alternatively at least about 90% amino acid sequence identity, alternatively at least about 91% amino acid sequence identity, alternatively at least about 92% amino acid sequence identity, alternatively at least about 93% amino acid sequence identity, alternatively at least about 94% amino acid sequence identity, alternatively at least about 95% amino acid sequence identity, alternatively at least about 968 amino acid sequence identity, alternatively at least about 97% amino acid sequence identity, alternatively at least about 98% amino acid sequence identity and alternatively at least about 99% amino acid sequence identity to the corresponding full-length native sequence. Ordinarily, variant polypeptides are at least about 10 amino acids in length, such as at least about 20 amino acids in length, e.g., at least about 30 amino acids in length, alternatively at least about 40 amino acids in length, alternatively at least about 50 amino acids in length, alternatively at least about 60 amino acids in length, alternatively at least about 70 amino acids in length, alternatively at least about 80 amino acids in length, alternatively at least about 90 amino acids in length, alternatively at least about 100 amino acids in length, alternatively at least about 150 amino acids in length, alternatively at least about 200 amino acids in length, alternatively at least about 300 amino acids in length, or more.

Particular variants include substitutions that are conservative in nature, i.e., those substitutions that take place within a family of amino acids that are related in their side chains. Specifically, amino acids are generally divided into four families: (1) acidic-aspartate and glutamate; (2) basic-lysine, arginine, histidine; (3) non-polar-alanine, valine, leucine, isoleucine, proline, phenylalanine, methionine, tryptophan; and (4) uncharged polar-glycine, asparagine, glutamine, cysteine, serine threonine, tyrosine. Phenylalanine, tryptophan, and tyrosine are sometimes classified as aromatic amino acids. For example, it is reasonably predictable that an isolated replacement of leucine with isoleucine or valine, an aspartate with a glutamate, a threonine with a serine, or a similar conservative replacement of an amino acid with a structurally related amino acid will not have a major effect on the biological activity. For example, the polypeptide of interest may include up to about 5-10 conservative or non-conservative amino acid substitutions, or even up to about 15-25 or 50 conservative or non-conservative amino acid substitutions, or any number between 5-50, so long as the desired function of the molecule remains intact.

By the term “degenerate variant” is intended for a polynucleotide containing changes in the nucleic acid sequence thereof, that encodes a polypeptide having the same amino acid sequence as the polypeptide encoded by the polynucleotide from which the degenerate variant is derived.

By “vector” is meant any genetic element, such as a plasmid, phage, transposon, cosmid, chromosome, virus, virion, etc., which is capable of replication when associated with the proper control elements and which can transfer gene sequences to cells. Thus, the term includes cloning and expression vehicles, as well as viral vectors. By “recombinant vector” is meant a vector that includes a heterologous nucleic acid sequence which is capable of expression in vivo.

For the purpose of describing the relative position of nucleotide sequences in a particular nucleic acid molecule throughout the instant application, such as when a particular nucleotide sequence is described as situated “upstream,” “downstream,” “3-prime (3′)” or “5-prime (5′)” relative to another sequence, it is to be understood that it is the position of the sequences in the “sense” or “coding” strand of a polynucleotide molecule that is being referred to as is conventional in the art.

The application of gene delivery strategies is frequently thwarted by inadequate systems for the delivery and expression of a transgene within target cell populations. In theory, the concept of gene delivery is simple: delivery of a “good gene” that will ultimately correct the defects of a disease. However, the execution of gene therapy is everything but simple. Indeed, the main problem of gene therapy is the lack of efficient, specific, and safe DNA delivery systems. This is particularly important in brain diseases, such as brain tumors or neurological syndromes caused by mutations in single genes. All current vectors cannot efficiently cross the blood-brain barrier to infect neural cells. Moreover, the fast-track advancement in genome editing and enzymatic tools to manipulate the DNA are simply useless in the absence of efficient ways to deliver therapeutic transgene to the brain. Provided herein is the development of a gene delivery system that utilizes a recombinantly modified Zika virus (ZIKV). It is expected that the ZIKV-based gene delivery system disclosed herein can be used both in vivo (even in utero to target the fetal brain), or in vitro, to infect hard-to-infect cells such as neurons and glia. The receptor that is targeted by the ZIKV-based gene delivery system disclosed herein, can be found in different cell types in the body. Thus, the ZIKV-based gene delivery system of the disclosure is an efficient tool for vertical transmission and infection of the developing brain and to cross the blood-brain barrier to infect the adult brain. In both cases, the efficiency is outstanding due to the dramatic neuro-tropism of ZIKV.

Zika virus (ZIKV) is a single-stranded positive-sense RNA arbovirus belonging to the Flavivirus genus of the Flaviviridae family, members of which cause widespread morbidity worldwide. It is spread by daytime active Aedes mosquitoes, such as A. aegypti and A. albopictus. Its name comes from the Zika Forest of Uganda, where the virus was first isolated in 1947. Zika virus shares a genus with the dengue, yellow fever, Japanese encephalitis, and West Nile viruses. Since the 1950s, it has been known to occur within a narrow equatorial belt from Africa to Asia. From 2007 to 2016, the virus spread eastward, across the Pacific Ocean to the Americas, leading to the 2015-2016 Zika virus epidemic.

The infection, known as Zika fever or Zika virus disease, often causes only mild symptoms, like a very mild form of dengue fever. While there is no specific treatment, acetaminophen and rest may help with the symptoms. No vaccines have been approved for clinical use, however, several vaccines are currently in clinical trials. Zika can spread from a pregnant person to their baby. This can result in microcephaly, severe brain malformations, and other birth defects. Zika infections in adults may result rarely in Guillain-Barre syndrome.

The ZIKV genome evolved rapidly from the Flavivirus genus and diverged from the members of this genus, even within the dengue virus cluster to which ZIKV belongs. Genome variations and divergences also exist among ZIKV strains/isolates. These genome divergences might account for the uniqueness of Zika disease. The ZIKV genome comprises an approximate 10.8-kb single-stranded positive-sense RNA molecule (SEQ ID NO: 1; FIG. 6, wherein T is U) that contains an ˜100 nt 5′ untranslated region (UTR) (SEQ ID NO: 2, wherein T is U), a single open reading frame of ˜10 kb, and an ˜420 nt 3′ UTR (SEQ ID NO: 13, wherein T is U). The open reading frame comprises the C-gene (SEQ ID NO: 3, wherein T is U), prM/M gene (SEQ ID NO: 4, wherein T is U), the E-gene (SEQ ID NO: 5, wherein T is U) and nonstructural protein coding sequences for NS1, NS2A, NS2B, NS3, NS4A, NS4B and NS5 (SEQ ID NOS: 6, 7, 8, 9, 10, 11 and 12, respectively, wherein T is U), thus encoding a single polyprotein, which is later processed into the capsid (C); the precursor membrane (prM); the envelope protein (E); and seven nonstructural proteins (NS1, NS2A, NS2B, NS3, NS4A, NS4B and NS5). The 5′ UTR contains the VRNA promoter and terminates with a type I cap, followed by the conserved dinucleotide AG. The 3′ end of the transcript is not polyadenylated and is instead terminated with a conserved CUOH. It will be recognized that various variants of the ZIKA virus are known and thus reference to any SEQ ID NO. above can be with reference to a percent identity while retaining functionality (e.g., sequences that are at least 85%, 87%, 90%, 92%, 95%, 98%, or 99% identical to any one of SEQ ID NOs: 1-13).

The C protein comprises the viral capsid, which is icosahedral in shape and surrounded by a spherical lipid bilayer membrane derived from the host. The M and E proteins are displayed on the viral surface and have transmembrane helices that anchor them in the outer membrane. As mature viral particles egress, the prM protein is cleaved by a furin-like protease located in the trans-Golgi network into the pr peptide and the M protein. It has been hypothesized that prior to this step, the prM protein participates in E protein folding. The E protein is the major virion surface protein and is involved in host cell binding and membrane fusion. The structure of the ZIKV E protein in complex with the flavivirus broadly neutralizing antibody 2A10G6 was also solved, and this antibody was shown to neutralize ZIKV infection in vitro and protect mice in vivo.

The nonstructural proteins (NS1-NS5) form the replicative complex and play a role in host innate immunity antagonism. The ZIKV NS1 protein is highly similar to other flaviviruses, with a noted divergent electrostatic potential on the loop surface. This divergent region is involved in binding host factors and protective antibodies, so it could be a potential antiviral target. The NS1 protein has also been implicated in immune evasion and appears to play a role in viral replication along with NS4A. The non-structural proteins NS2A, NS2B, NS4A, and NS4B are hydrophobic proteins that may be membrane-associated. However, they do not have any known enzymatic motifs, and their specific functions have yet to be elucidated. The NS3 protein is central to viral replication and polyprotein processing due to its N-terminal protease domain along with its C-terminal RNA helicase activity. The NS5 protein has two known activities: an RNA-dependent RNA polymerase activity performed by the C terminus and an RNA capping function executed by the methyltransferase domain located at the N terminus. NS5 suppresses IFN signaling via proteasome-dependent degradation of human STAT2. In a particular embodiment, the recombinant Zika-based vector of the disclosure is noncompetent in that it cannot form infectious particles in targeted cells.

A recombinantly modified ZIKV genome useful in the methods and compositions of the disclosure comprises the 5′ UTR and 3′ UTR of the ZIKV genome and genes for nonstructural proteins NS2A, NS2B, NS3, NS4A, NS4B, and/or NSS5. In a further embodiment, the recombinantly modified ZIKV genome does not comprise one or more genes for protein C, prM/M, and/or protein E or comprise truncated genes encoding protein C, prm/M, protein E, and any combination thereof. In yet a further embodiment, the recombinantly modified ZIKV genome comprises a portion of the gene for NS1. In a further embodiment, the recombinantly modified ZIKV genome further comprises a heterologous gene or transgene that is inserted into the ZIKV genome after the 5′ UTR but prior to the genes for the nonstructural proteins. In yet a further embodiment, the heterologous gene or transgene is used (1) to prevent, treat, or ameliorate a genetic disorder, including a genetic neurodegenerative disorder; (2) to replace mutated genes (e.g., defective p53 gene); (3) to fix mutated genes; and (4) to make diseased cells (e.g., cancerous cells) more evident to the immune system. In yet further embodiments, more than one transgene encoding more than one of the therapeutic molecules may be used with the recombinantly modified ZIKV-based particle or vectors disclosed herein, wherein each transgene is operably linked to a control element to enable the expression of the transgenes from a single recombinantly modified ZIKV-based particle or vector disclosed herein. In additional methods, the transgenes may be operably linked to the same regulatory element. Each transgene encodes a biologically active molecule, expression of which in the targeted tissue results in at least partial correction of a disease or disorder. Additionally, in cases where more than one transgene is delivered, the transgenes may be delivered via more than one recombinantly modified ZIKV-based particle or vector disclosed herein, wherein each ZIKV-based particle or vector comprises a transgene operably linked to a promoter. Examples of heterologous genes or transgenes that can be used with the recombinantly modified ZIKV-based particles or vectors disclosed herein are described throughout the present document and include but are not limited to, EGFP, MECP2, antisense oligonucleotides (ASOs), and a CRISPR/Cas enzyme.

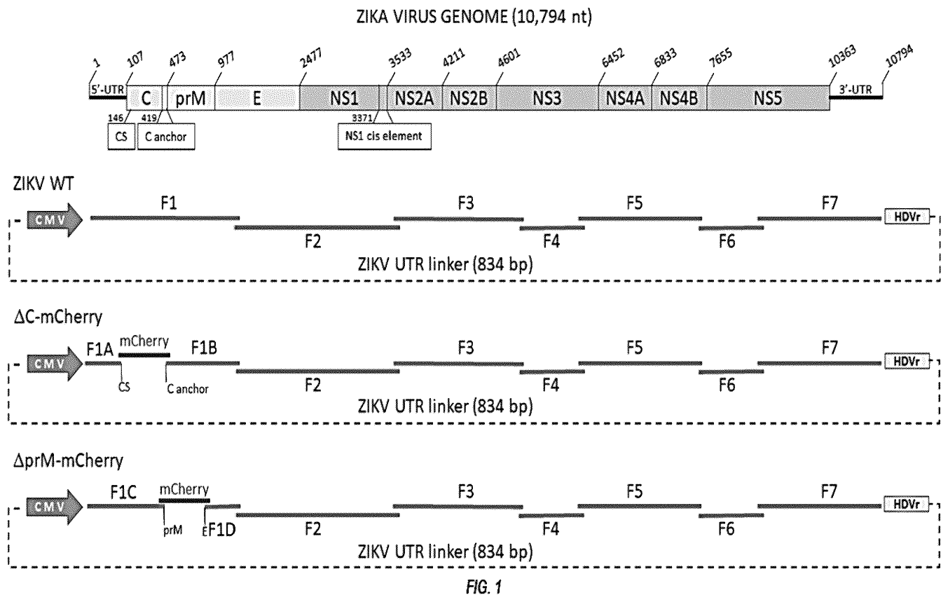

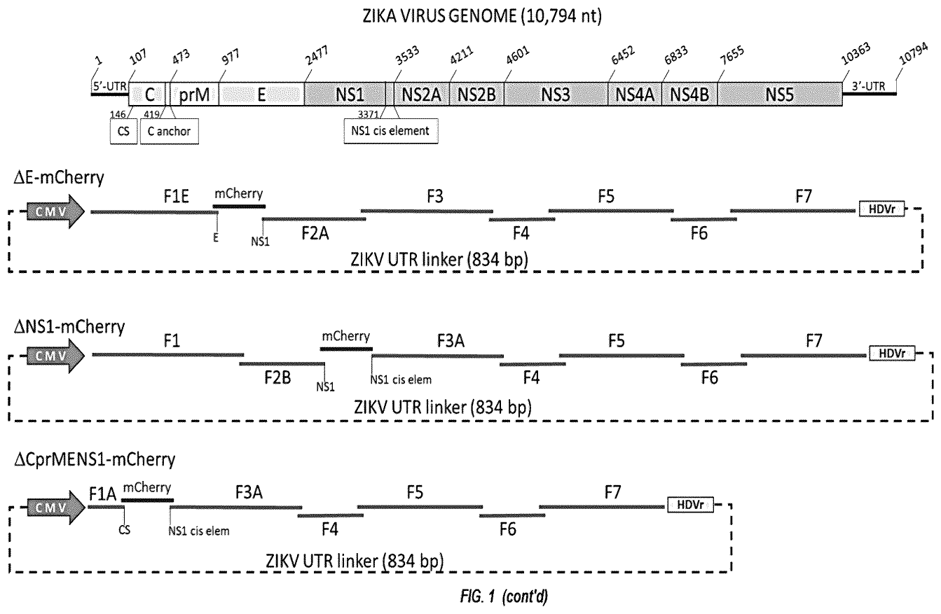

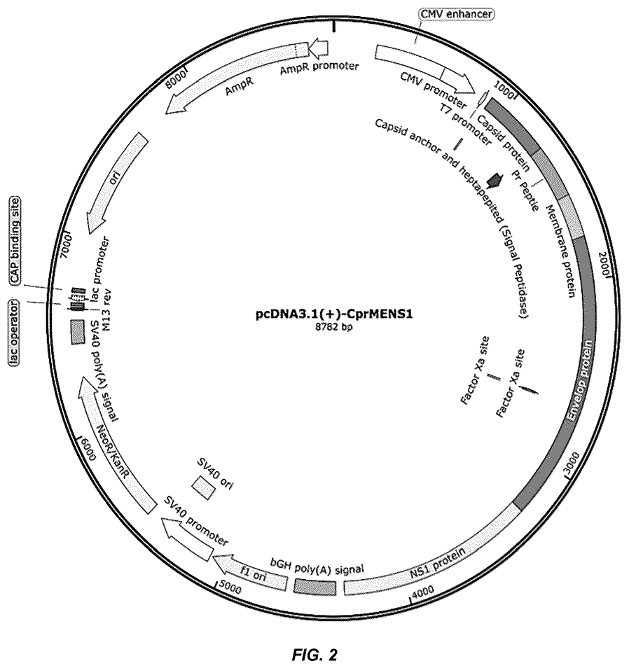

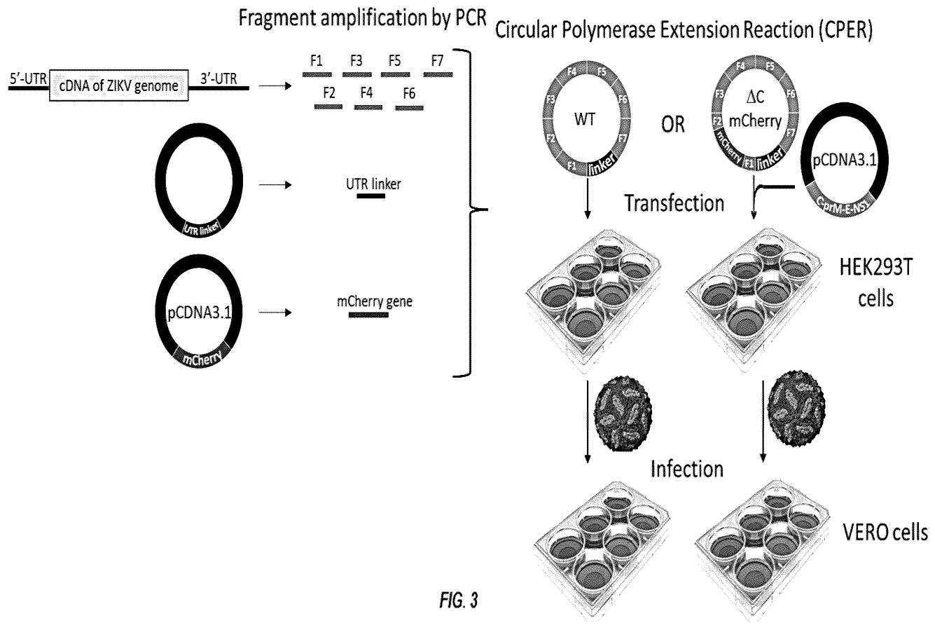

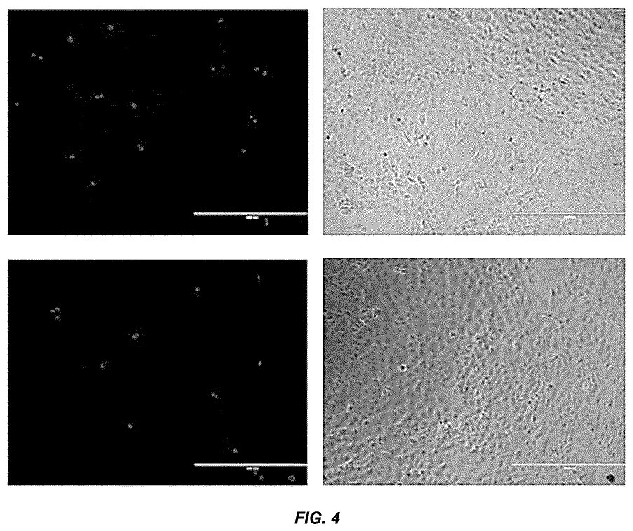

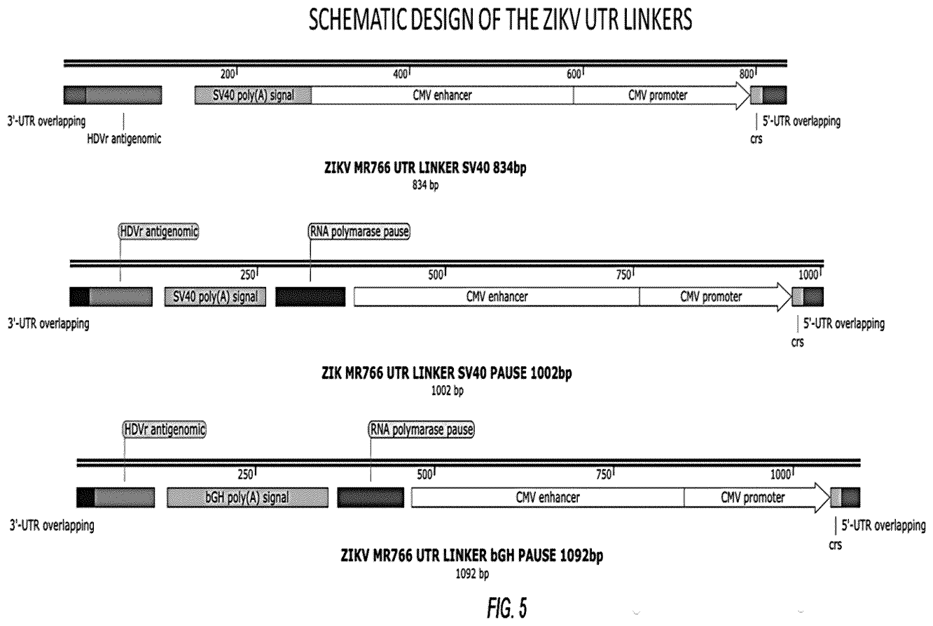

Provided herein are recombinant ZIKV genomes and recombinantly modified ZIKV particles carrying heterologous polynucleotide(s) for efficient delivery in target cells (e.g. immature and mature brain cells). As shown in the studies presented herein, ZIKV particles were engineered to carry different reporter genes (e.g., mCherry) in the place of certain (or a plurality of) viral genes, such as the prM using the Circular Polymerase Extension Reaction (CPER) approach. CPER is a versatile alternative for infectious clones. In the CPER approach, several DNA fragments amplified from the viral genome with overlapping ends in the contiguous fragments were assembled by a polymerization reaction in the presence of another recombinant fragment containing in frame an overlapping DNA sequence of the 3′-UTR region of the virus genome, HDVr antigenomic sequence, SV40 or bGH (polyA) signal, CMV enhancer, CMV promoter, RNA polymerase pause sequence and an overlapping DNA sequence of the 5′-UTR region of the virus genome (e.g., see FIG. 1). The PCR containing all these fragments produced a circularized DNA which was transfected into HEK293T by lipofectamine LTX (Thermofisher). After 3 days, the supernatant was found to contain infectious viral particles that were the same as wild-type ZIKV. The CPER approach was very advantageous because the fragments can be easily replaced by another gene containing overlapping DNA sequences to the contiguous fragments. To produce attenuated ZIKV particles carrying one or more heterologous polynucleotides, only structural proteins (C, prM/M and E) and part of NS1 protein should be replaced because they cannot form infectious particles in the target cells, but RNA virus replication function should be maintained. The substituted gene was provided by trans-complementation method so that the non-infectious viral particles were assembled in vitro (e.g., see FIG. 3). As a proof-of-concept, the supernatant of the recombinant ZIKV carrying the mCherry reporter gene in substitution of prM gene sequence was used to transfect VERO E6 cells (e.g., see FIG. 4). The study confirmed that the non-replicative, recombinant ZIKV-derived vector transduced a reporter gene into human cells.

A recombinantly modified Zika virus (ZIKV)-based particle or vector, comprising an RNA genome having from 5′ to 3′: a 5′ UTR from a Flavivirus (such as SEQ ID NO:2 or sequences that are at least 85% identical thereto and wherein T is U); one or more heterologous genes comprising a transgene cassette inserted into or replacing coding sequences for genes selected from the group consisting of a capsid gene (C gene) (SEQ ID NO: 3, wherein T is U or a sequence that is at least 85% identical thereto), a pRM/M gene (SEQ ID NO:4, wherein T is U or a sequence that is at least 85% identical thereto), an envelope gene (E gene) (SEQ ID NO: 5, wherein T is U or a sequence that is at least 85% identical thereto), an NS1 gene (SEQ ID NO: 6, wherein T is U or a sequence that is at least 85% identical thereto), and any combination thereof of two consecutive coding sequences of a ZIKA virus genome; one or more genes for nonstructural proteins NS1, NS2A, NS2B, NS3, NS4A, NS4B, and/or NS5 from ZIKV (e.g., SEQ ID NOs: 6-12, wherein T is U or a sequence that is at least 858 identical thereto; and a 3′ UTR from a Flavivirus (e.g., SEQ ID NO: 13, wherein T is U or a sequence that is at least 85% identical thereto). In certain embodiments, the vector comprises a genome that lacks the genes for protein C, prM/M, and/or protein E from ZIKV, or wherein the vector does not produce protein C, prM/M, and/or protein E from ZIKV. In such embodiments, one of the C-gene; prM/M gene; E-gene; C- and prM/M gene; C-, prM/M-, and E-gene; prM/M- and E-gene are disrupted by one or more heterologous polynucleotides. In another embodiment, the vector comprises a genome that has a 5′ UTR, 3′UTR, at least one heterologous polynucleotide in a transgene cassette and coding sequences for NS2A, NS2B, NS3, NS4A, NS4B and NS5 (e.g., SEQ ID NOs: 7-12, wherein T is U, or sequences at least 85% identical thereto). In another embodiment, the transgene cassette is about 50 bp to 4 kb in size.

The disclosure provides a recombinant ZIKV genome or ZIKV-based particle or vector that contains a ZIKV genome comprising an RNA genome, the RNA genome comprising from 5′ to 3′: a 5′ cap; a 5′ UTR having a sequence that is at least 85% identical to SEQ ID NO: 2, wherein T is U; a transgene cassette replacing nucleotides 61 to 312 of the capsid gene (SEQ ID NO: 3, wherein T is U, or a sequence that is at least 858, 908, 958, 98% or 99% identical thereto); a prM/M coding sequence (SEQ ID NO: 4, wherein T is U, or a sequence that is at least 858, 908, 958, 98% or 99% identical thereto); an E protein coding sequence (SEQ ID NO: 5, wherein T is U, or a sequence that is at least 858, 908, 958, 98% or 99% identical thereto; non-structural protein coding sequences of NS1, NS2A, NS2B, NS3, NS4A, NS4B and NS5 (SEQ ID NO: 6-12, respectively, wherein T is U, or a sequence that is at least 858, 908, 958, 98% or 998 identical thereto); a 3′ UTR (SEQ ID NO: 13, wherein T is U, or a sequence that is at least 858, 908, 958, 98% or 99% identical thereto); wherein the transgene cassette is operably linked to a 5′UTR or comprises an internal ribosome binding site; and optionally cis-acting sequences necessary for packaging and replication in a target cell.

The disclosure provides a recombinant ZIKV genome or ZIKV-based particle or vector that contains a ZIKV genome comprising an RNA genome, the RNA genome comprising from 5′ to 3′: a 5′ cap; a 5′ UTR (SEQ ID NO: 2, wherein T is U, or a sequence that is at least 858, 90%, 958, 98% or 99% identical thereto); a capsid coding sequence (SEQ ID NIO: 3, wherein T is U, or a sequence that is at least 858, 908, 958, 98% or 99% identical thereto); a transgene cassette replacing nucleotides 16 to 486 of the prM/M gene (SEQ ID NO: 4, wherein T is U, or a sequence that is at least 858, 908, 95%, 98% or 99% identical thereto); an E protein coding sequence (SEQ ID NO: 5, wherein T is U, or a sequence that is at least 858, 908, 95%, 98% or 99% identical thereto); non-structural protein coding sequences of NS1, NS2A, NS2B, NS3, NS4A, NS4B and NS5 (SEQ ID NO: 6-12, respectively, wherein T is U, or a sequence that is at least 85%, 90%, 958, 98% or 99% identical thereto); a 3′ UTR (SEQ ID NO: 13, wherein T is U, or a sequence that is at least 858, 908, 95%, 98% or 99% identical thereto); wherein the transgene cassette is operably linked to a 5′UTR or comprises an internal ribosome binding site; and optionally cis-acting sequences necessary for packaging and replication in a target cell.

The disclosure provides a recombinant ZIKV genome or ZIKV-based particle or vector that contains a ZIKV genome comprising an RNA genome, the RNA genome comprising from 5′ to 3′: a 5′ cap; a 5′ UTR (SEQ ID NO: 2, wherein T is U, or a sequence that is at least 85%, 90%, 958, 98% or 99% identical thereto); a capsid coding sequence (SEQ ID NO: 3, wherein T is U, or a sequence that is at least 858, 908, 958, 98% or 99% identical thereto); a prM/M coding sequence (SEQ ID NO: 4, wherein T is U, or a sequence that is at least 858, 908, 958, 98% or 998 identical thereto); a transgene cassette replacing nucleotides 16 to 1494 of the Envelope gene (SEQ ID NO: 5, wherein T is U, or a sequence that is at least 858, 908, 95%, 98% or 99% identical thereto); non-structural protein coding sequences of NS1, NS2A, NS2B, NS3, NS4A, NS4B and NS5 (SEQ ID NO: 6-12, respectively, wherein T is U, or a sequence that is at least 858, 908, 958, 98% or 99% identical thereto); a 3′ UTR; wherein the transgene cassette is operably linked to a 5′UTR or comprises an internal ribosome binding site; and optionally cis-acting sequences necessary for packaging and replication in a target cell.

The disclosure provides a recombinant ZIKV genome or ZIKV-based particle or vector that contains a ZIKV genome comprising an RNA genome, the RNA genome comprising from 5′ to 3′: a 5′ cap; a 5′ UTR (SEQ ID NO: 2, wherein T is U, or a sequence that is at least 85%, 90%, 958, 98% or 99% identical thereto); a capsid coding sequence (SEQ ID NO: 3, wherein T is U, or a sequence that is at least 858, 908, 958, 98% or 99% identical thereto); a prM/M coding sequence (SEQ ID NO: 4, wherein T is U, or a sequence that is at least 858, 908, 958, 98% or 99% identical thereto); an E protein coding sequence (SEQ ID NO:5, wherein T is U, or a sequence that is at least 858, 908, 958, 98% or 99% identical thereto); a transgene cassette replacing nucleotides 16 to 894 of the NS1 gene (SEQ ID NO: 6, wherein T is U, or a sequence that is at least 858, 908, 958, 98% or 99% identical thereto); non-structural protein coding sequences of NS2A, NS2B, NS3, NS4A, NS4B and NS5 (SEQ ID NO: 7-12, respectively, wherein T is U, or a sequence that is at least 85%, 908, 958, 98% or 99% identical thereto); a 3′ UTR (SEQ ID NO: 13, wherein T is U, or a sequence that is at least 858, 908, 958, 98% or 99% identical thereto); wherein the transgene cassette is operably linked to a 5′UTR or comprises an internal ribosome binding site; and optionally cis-acting sequences necessary for packaging and replication in a target cell.

The disclosure provides a recombinant ZIKV genome or ZIKV-based particle or vector that contains a ZIKV genome comprising an RNA genome, the RNA genome comprising from 5′ to 3′: a 5′ cap; a 5′ UTR (SEQ ID NO: 2, wherein T is U, or a sequence that is at least 858, 908, 958, 98% or 99% identical thereto); a transgene cassette replacing nucleotides beginning at nucleotide 61 of SEQ ID NO: 3 (the capsid gene) to nucleotide 894 of SEQ ID NO: 6 (the NS1 gene); non-structural protein coding sequences of NS2A, NS2B, NS3, NS4A, NS4B and NS5 (SEQ ID NO: 7-12, respectively, wherein T is U, or a sequence that is at least 858, 908, 958, 98% or 99% identical thereto); a 3′ UTR (SEQ ID NO: 13, wherein T is U, or a sequence that is at least 85%, 908, 958, 98% or 998 identical thereto); wherein the transgene cassette is operably linked to a 5′UTR or comprises an internal ribosome binding site; and optionally cis-acting sequences necessary for packaging and replication in a target cell.

The recombinantly modified ZIKV particles disclosed herein can be transmitted horizontally and vertically. Efficient transmission of the recombinantly modified ZIKV particles is generally dependent upon the expression on the target cell of receptors that specifically recognize the ZIKV viral envelope proteins, although ZIKV may use receptor-independent, nonspecific routes of entry at low efficiency.

In another embodiment, a targeting polynucleotide sequence is included as part of the recombinantly modified ZIKV-based vector or particle of the disclosure. The targeting polynucleotide sequence is a targeting ligand (e.g., peptide hormones such as heregulin, a single-chain antibodies, a receptor, or a ligand for a receptor), a tissue-specific or cell-type specific regulatory element (e.g., a tissue-specific or cell-type specific promoter or enhancer), or a combination of a targeting ligand and a tissue-specific/cell-type specific regulatory element. The recombinantly modified ZIKV-based vector or particle of the disclosure is therefore genetically modified in such a way that the virus is targeted to a particular cell type (e.g., neural cells, glial cells, astrocytes, smooth muscle cells, hepatic cells, renal cells, fibroblasts, keratinocytes, mesenchymal stem cells, bone marrow cells, chondrocyte, epithelial cells, intestinal cells, neoplastic cells, glioma cells, neuronal cells and others known in the art). The first way directs the ZIKV-based vector or particle to a target cell by binding to cells having a molecule on the external surface of the cell. This method of targeting the ZIKV-based vector or particle utilizes the expression of a targeting ligand to assist in targeting the ZIKV-based vector or particle to cells or tissues that have a receptor or binding molecule that interacts with the targeting ligand. By inserting a heterologous nucleic acid sequence of interest into the ZIKV-based vector or particle of the disclosure, along with another gene that encodes, for example, the ligand for a receptor on a specific target cell, the ZIKV-based vector or particle is now target-specific. Typically, the targeting domain will be operably linked to the endogenous gene for the ZIKA virus associated with targeting, thereby providing a chimeric polypeptide. Viral vectors can be made target-specific by attaching, for example, a sugar, a glycolipid, or a protein. Targeting can be accomplished by using an antibody to target the ZIKV-based vector or particle. Those of skill in the art will know of, or can readily ascertain, specific polynucleotide sequences that can be inserted into the ZIKV-based genome or proteins that can be attached to a ZIKV-based envelope to allow target-specific delivery of the ZIKV-based vector or particle vector containing the nucleic acid sequence of interest.

The recombinantly modified ZIKV-based vectors or particles of the disclosure can be used in gene delivery applications to prevent, treat, or ameliorate a genetic disorder. Genetic disorders caused by recessive null mutations represent the most straightforward group as the replacement of the missing wild-type protein by using the recombinantly modified ZIKV-based particles of the disclosure should prove effective in rescuing the disorder phenotype. If the causative gene for a rare monogenic disease has not been identified or the mode of inheritance is complex as in late-onset neurodegenerative diseases, use of recombinantly modified ZIKV-based particles of the disclosure can still be contemplated as a gene therapy strategy. The approach in these situations involve transfecting the cell with heterologous gene or transgenes that upregulate the expression of trophic factors, which in turn serve to rescue neuronal cells from impending death or at least prolong their survival. These blanket neuroprotective strategies could also be used to supplement more targeted gene therapy in monogenic diseases and conceptually, these could provide a synergistic beneficial effect. An alternative approach for neurodegenerative diseases is optogenetics, which involves the introduction of light-sensitive protein sensors into neurons using recombinantly modified ZIKV-based particles of the disclosure to make the neurons functionally photosensitive. Ion channel proteins of the channelrhodopsin, halorhodopsin and archaerhodopsins families can confer these unique properties by modulating neuronal membrane potential and the balance between depolarized and hyperpolarized states. Optogenetics is being used to convert non-photosensitive retinal cells into artificial photoreceptors, and to deliberately switch on and off specific central nervous system pathways to circumvent the damaged circuitry in anatomically diseased area.

As the recombinantly modified ZIKV-based particles disclosed herein can cross the blood-brain barrier, the use of the ZIKV-based particles of the disclosure are ideally suited for delivering genes to the cells of the brain. In a particular embodiment, the compositions and/or method of the disclosure are used to prevent, treat, or ameliorate a genetic disorder. In a further embodiment, the genetic disorder is a genetic neurodegenerative disorder. Examples of genetic neurodegenerative disorders include but are not limited to, Alzheimer's disease, amyotrophic lateral sclerosis (ALS), prion disease, Parkinson's disease, Leber congenital amaurosis (LCA), choroideremia, Leber hereditary optic neuropathy (LHON), and Huntington's disease.

In another embodiment, the disclosure provides a method of treating a subject having a genetic disorder using the recombinantly modified ZIKV-based particles or vectors disclosed herein. The subject can be any mammal, and is preferably a human. The subject is contacted with a recombinantly modified ZIKV-based particles or vectors of the disclosure. The contacting can be in vivo or ex vivo. Methods of administering the recombinantly modified ZIKV-based particles or vectors of the disclosure are known in the art and include, for example, systemic administration, topical administration, intraperitoneal administration, intra-muscular administration, intracranial, cerebrospinal, as well as administration directly at the site of a genetic disorder. In a particular embodiment, the recombinantly modified ZIKV-based particles or vectors of the disclosure are administered by systemic peripheral injection.

Thus, the disclosure includes various pharmaceutical compositions useful for treating a genetic disorder. The pharmaceutical compositions, according to the disclosure, are prepared by bringing recombinantly modified ZIKV-based particles or vectors comprising a heterologous polynucleotide or transgene useful for preventing, treating, modulating, or ameliorating a genetic disorder according to the disclosure into a form suitable for administration to a subject using carriers, excipients and additives or auxiliaries. Frequently used carriers or auxiliaries include magnesium carbonate, titanium dioxide, lactose, mannitol and other sugars, talc, milk protein, gelatin, starch, vitamins, cellulose and its derivatives, animal and vegetable oils, polyethylene glycols and solvents, such as sterile water, alcohols, glycerol and polyhydric alcohols. Intravenous vehicles include fluid and nutrient replenishers. Preservatives include antimicrobials, anti-oxidants, chelating agents, and inert gases. Other pharmaceutically acceptable carriers include aqueous solutions non-toxic excipients, including salts, preservatives, buffers, and the like, as described, for instance, in Remington's Pharmaceutical Sciences, 15th ed. Easton: Mack Publishing Co., 1405-1412, 1461-1487 (1975) and The National Formulary XIV., 14th ed. Washington: American Pharmaceutical Association (1975), the contents of which are hereby incorporated by reference. The pH and exact concentration of the various components of the pharmaceutical composition are adjusted according to routine skills in the art. See Goodman and Gilman's The Pharmacological Basis for Therapeutics (7th ed.).

Methods of making a recombinant ZIKA-based particle or vector comprises expressing a vector comprising a plasmid backbone of SEQ ID NO: 15 (pBR322), wherein the infection clone is inserted between base-pair 38 and 39 of SEQ ID NO: 15. The infectious clone will have the general sequences and constructs identified elsewhere herein, but will comprise a CMV enhancer, promoter and crs sequences (SEQ ID NO: 17) upstream of the 5′UTR, and HDVr antigenomic and SV40 (SEQ ID NO: 18) (or bGH (SEQ ID NO: 19)) (polyA) sequences downstream of the 3′UTR. In some instances an HDVr antigenomic, SV40 (polyA) and RNA polymerase pause sequence (SEQ ID NO: 20) are located downstream of the 3′UTR. In other embodiments, an HDVr antigenomic, bGH (polyA), and RNA polymerase pause sequences (SEQ ID NO: 21) are located downstream of the 3′-UTR sequence. An exemplary infectious clone (ZIKV MR766) SEQ ID NO: 16 is provided. In still further embodiments, to make the infectious clone less toxic to the host cell (e.g., E. coli) a chimeric intron sequence comprises of human β-globin and immunoglobulin heavy chain (SEQ ID NO:22) was inserted into different locations of the ZIKV MR766 coding sequence (SEQ ID NO: 16). For example, the chimeric intron can be inserted between the bases 639 and 640 of the NS1 gene and/or between the bases 1155 and 1156 of the NS5 gene. The recombinant plasmid is then closed into a suitable host cell for expression (e.g., HEK293T cells).

As previously discussed, general texts which describe molecular biological techniques useful herein, including the use of vectors, promoters, and many other relevant topics, include Berger and Kimmel, Guide to Molecular Cloning Techniques, Methods in Enzymology Volume 152, (Academic Press, Inc., San Diego, Calif.) (“Berger”); Sambrook et al., Molecular Cloning—A Laboratory Manual, 2d ed., Vol. 1-3, Cold Spring Harbor Laboratory, Cold Spring Harbor, N.Y., 1989 (“Sambrook”) and Current Protocols in Molecular Biology, F. M. Ausubel et al., eds., Current Protocols, a joint venture between Greene Publishing Associates, Inc. and John Wiley & Sons, Inc., (supplemented through 1999) (“Ausubel”). Examples of protocols sufficient to direct persons of skill through in vitro amplification methods, including the polymerase chain reaction (PCR), the ligase chain reaction (LCR), QB-replicase amplification, and other RNA polymerase mediated techniques (e.g., NASBA), e.g., for the production of the homologous nucleic acids of the disclosure are found in Berger, Sambrook, and Ausubel, as well as in Mullis et al. (1987) U.S. Pat. No. 4,683,202; Innis et al., eds. (1990) PCR Protocols: A Guide to Methods and Applications (Academic Press Inc. San Diego, Calif.) (“Innis”); Arnheim & Levinson (Oct. 1, 1990) C&EN 36-47; The Journal Of NIH Research (1991) 3:81-94; Kwoh et al. (1989) Proc. Natl. Acad. Sci. USA 86:1173; Guatelli et al. (1990) Proc. Nat'l. Acad. Sci. USA 87:1874; Lomell et al. (1989) J. Clin. Chem 35:1826; Landegren et al. (1988) Science 241:1077-1080; Van Brunt (1990) Biotechnology 8:291-294; Wu and Wallace (1989) Gene 4:560; Barringer et al. (1990) Gene 89:117; and Sooknanan and Malek (1995) Biotechnology 13:563-564. Improved methods for cloning in vitro amplified nucleic acids are described in Wallace et al., U.S. Pat. No. 5,426,039. Improved methods for amplifying large nucleic acids by PCR are summarized in Cheng et al. (1994) Nature 369:684-685 and the references cited therein, in which PCR amplicons of up to 40 kb are generated. One of skill in the art will appreciate that essentially any RNA can be converted into a double stranded DNA suitable for restriction digestion, PCR expansion and sequencing using reverse transcriptase and a polymerase. See, e.g., Ausubel, Sambrook and Berger, all supra.

A number of embodiments of the disclosure been described. Nevertheless, it will be understood that various modifications may be made without departing from the spirit and scope of the disclosure. Accordingly, other embodiments are within the scope of the following claims.

1. A recombinantly modified Zika virus (ZIKV)-based particle or vector, comprising:
an RNA genome having from 5′ to 3′:
a 5′ UTR from a Flavivirus;
one or more heterologous genes comprising a transgene cassette inserted into or replacing coding sequences for genes selected from the group consisting of a capsid gene (C gene), a pRM/M gene, an envelope gene (E gene), an NS1 gene, and any combination thereof;
one or more genes for nonstructural proteins NS1, NS2A, NS2B, NS3, NS4A, NS4B, and/or NS5 from ZIKV; and
a 3′ UTR from a Flavivirus.

2. The recombinantly modified ZIKV-based particle or vector of claim 1, wherein the ZIKV-based particle or vector does not comprise the genes for protein C, prM/M, and/or protein E from ZIKV.

3. The recombinantly modified ZIKV-based particle or vector of claim 1, wherein the 3′ UTR is not polyadenylated and is terminated with CU_{OH}.

4. (canceled)

5. The recombinantly modified ZIKV-based particle or vector of claim 1, wherein the ZIKV-based particle or vector comprises genes for NS2A, NS2B, NS3, NS4A, NS4B, and NS5 from ZIKV.

6. The recombinantly modified ZIKV-based particle or vector of claim 1, comprising:
a capsid;
an RNA genome, the RNA genome comprising from 5′ to 3′:
a 5′ cap;
a 5′ UTR;
a transgene cassette replacing nucleotides 61 to 312 of the capsid gene;
a prM/M coding sequence;
an E protein coding sequence;
non-structural protein-coding sequences of NS1, NS2A, NS2B, NS3, NS4A, NS4B and NS5;
a 3′ UTR;
wherein the transgene cassette is operably linked to a 5′UTR or comprises an internal ribosome binding site; and
cis-acting sequences necessary for packaging and replication in a target cell.

7. The recombinantly modified ZIKV-based particle or vector of claim 1 comprising:
a capsid;
an RNA genome, the RNA genome comprising from 5′ to 3′:
a 5′ cap;
a 5′ UTR;
a capsid coding sequence
a transgene cassette replacing nucleotides 16 to 486 of the prM/M gene;
an E protein coding sequence;
non-structural protein coding sequences of NS1, NS2A, NS2B, NS3, NS4A, NS4B and NS5;
a 3′ UTR;
wherein the transgene cassette is operably linked to a 5′UTR or comprises an internal ribosome binding site; and
cis-acting sequences necessary for packaging and replication in a target cell.

8. The recombinantly modified ZIKV-based particle or vector of claim 1 comprising:
a capsid;
an RNA genome, the RNA genome comprising from 5′ to 3′:
a 5′ cap;
a 5′ UTR;
a capsid coding sequence;
a prM/M coding sequence;
a transgene cassette replacing nucleotides 16 to 1494 of the Envelope gene;
non-structural protein coding sequences of NS1, NS2A, NS2B, NS3, NS4A, NS4B and NS5;
a 3′ UTR;
wherein the transgene cassette is operably linked to a 5′UTR or comprises an internal ribosome binding site; and
cis-acting sequences necessary for packaging and replication in a target cell.

9. The recombinantly modified ZIKV-based particle or vector of claim 1 comprising:
a capsid;
an RNA genome, the RNA genome comprising from 5′ to 3′:
a 5′ cap;
a 5′ UTR;
a capsid coding sequence;
a prM/M coding sequence;
an E protein coding sequence;
a transgene cassette replacing nucleotides 16 to 894 of the NS1 gene;
non-structural protein coding sequences of NS2A, NS2B, NS3, NS4A, NS4B and NS5;
a 3′ UTR;
wherein the transgene cassette is operably linked to a 5′UTR or comprises an internal ribosome binding site; and
cis-acting sequences necessary for packaging and replication in a target cell.

10. The recombinantly modified ZIKV-based particle or vector of claim 1 comprising:
a capsid;
an RNA genome, the RNA genome comprising from 5′ to 3′:
a 5′ cap;
a 5′ UTR;
a transgene cassette replacing nucleotides beginning at nucleotide 61 of the capsid gene to nucleotide 894 of the NSA gene;
non-structural protein-coding sequences of NS2A, NS2B, NS3, NS4A, NS4B and NS5;
a 3′ UTR;
wherein the transgene cassette is operably linked to a 5′UTR or comprises an internal ribosome binding site and
cis-acting sequences necessary for packaging and replication in a target cell.

11.-13. (canceled)

14. The recombinantly modified ZIKV-based particle or vector of claim 1, wherein the ZIKV-based particle or vector comprises more than one heterologous gene in the transgene cassette, wherein each heterologous gene is operably linked to a regulatory element.

15. (canceled)

16. The recombinantly modified ZIKV-based particle or vector of claim 14, wherein each heterologous gene or transgene is operably linked to different regulatory elements.

17. The recombinant modified ZIKV-based particle or vector of claim 14, wherein each heterologous gene is separated by a P2A self-cleavable linker coding domain.

18. The recombinant modified ZIKV-based particle or vector of claim 1, wherein the RNA genome is engineered from a polynucleotide having a sequence that is at 98% identical to SEQ ID NO:1, wherein T is U.

19. The recombinant modified ZIKV-based particle or vector of claim 1, wherein the transgene cassette is from 30 to 4000 bp.

20. The recombinant modified ZIKV-based particle or vector of claim 14, wherein the regulatory element comprises an internal ribosome entry site (IRES).

21. The recombinant modified ZIKV-based particle or vector of claim 1, wherein
the 5′UTR comprises a sequence that is at least 98% identical to SEQ ID NO: 2, wherein T is U;
the capsid gene comprises a sequence that is at least 98% identical to SEQ ID NO: 3, wherein T is U;
the prM/M gene comprises a sequence that is at least 98% identical to SEQ ID NO: 4, wherein T is U;
the envelop gene comprises a sequence that is at least 98% identical to SEQ ID NO: 5, wherein T is U;
the NS1 gene comprises a sequence that is at least 98% identical to SEQ ID NO: 6, wherein T is U;
the NS2A gene comprises a sequence that is at least 98% identical to SEQ ID NO: 7, wherein T is U;
the NS2B gene comprises a sequence that is at least 98% identical to SEQ ID NO: 8, wherein T is U;
the NS3 gene comprises a sequence that is at least 98% identical to SEQ ID NO: 9, wherein T is U:
the NS4A gene comprises a sequence that is at least 98% identical to SEQ ID NO: 10, wherein T is U;
the NS4B gene comprises a sequence that is at least 98% identical to SEQ ID NO: 11, wherein T is U;
the NS5 gene comprises a sequence that is at least 98% identical to SEQ ID NO: 12, wherein T is U; and/or
the 3′UTR comprises a sequence that is at least 98% identical to SEQ ID NO: 13, wherein T is U.

22.-32. (canceled)

33. The recombinant modified ZIKV-based particle or vector of claim 1, wherein the heterologous gene encodes a biological response modifier or an immunopotentiating cytokine.

34. The recombinant modified ZIKV-based particle or vector of claim 33, wherein the immunopotentiating cytokine is selected from the group consisting of interleukins 1 through 38, interferon, tumor necrosis factor (TNF), and granulocyte-macrophage-colony stimulating factor (GM-CSF).

35. The recombinant modified ZIKV-based particle or vector of claim 33, wherein the immunopotentiating cytokine is interferon gamma.

36. The recombinant modified ZIKV-based particle or vector of claim 1, wherein the heterologous gene encodes a polypeptide that converts a nontoxic prodrug into a toxic drug.

37. The recombinant modified ZIKV-based particle or vector of claim 36, wherein the polypeptide that converts a nontoxic prodrug into a toxic drug is thymidine kinase, purine nucleoside phosphorylase (PNP), or cytosine deaminase.

38. The recombinant modified ZIKV-based particle or vector of claim 1, wherein the heterologous gene encodes a receptor domain, an antibody, or an antibody fragment.

39. The recombinant modified ZIKV-based particle or vector of claim 1, wherein the heterologous gene comprises an inhibitory polynucleotide.

40. The recombinant modified ZIKV-based particle or vector of claim 39, wherein the inhibitory polynucleotide comprises a miRNA, RNAi or siRNA sequence.

41. A recombinant polynucleotide for producing the recombinant modified ZIKV-based particle or vector of claim 1.

42. A method of delivering a gene or polynucleotide to a cell comprising contacting the cell with the recombinant modified ZIKV-based particle or vector of claim 1 under conditions such that the heterologous polynucleotide is expressed.


SEQUENCE LISTING (22 sequences)

SEQ ID NO: 1 - DNA, 10799 bp, synthetic construct
  source 1..10799: mol_type = other DNA
atctgtgtgagtcagactgcgacagttcgagtctgaagcgagagctaacaacagtatcaacaggtttaatttggatttggaaacgagagtttctggtcatgaaaaacccaaagaagaaatccggaggattccggattgtcaatatgctaaaacgcggagtagcccgtgtaaaccccttgggaggtttgaagaggttgccagccggacttctgctgggtcatggacccatcagaatggttttggcgatactagcctttttgagatttacagcaatcaagccatcactgggccttatcaacagatggggttccgtggggaaaaaagaggctatggaaataataaagaagttcaagaaagatcttgctgccatgttgagaataatcaatgctaggaaagagaggaagagacgtggcgcagacaccagcatcggaatcattggcctcctgctgactacagccatggcagcagagatcactagacgcgggagtgcatactacatgtacttggataggagcgatgccgggaaggccatttcgtttgctaccacattgggagtgaacaagtgccacgtacagatcatggacctcgggcacatgtgtgacgccaccatgagttatgagtgccctatgctggatgagggagtggaaccagatgatgtcgattgctggtgcaacacgacatcaacttgggttgtgtacggaacctgtcatcacaaaaaaggtgaggcacggcgatctagaagagccgtgacgctcccttctcactctacaaggaagttgcaaacgcggtcgcagacctggttagaatcaagagaatacacgaagcacttgatcaaggttgaaaactggatattcaggaaccccgggtttgcgctagtggccgttgccattgcctggcttttgggaagctcgacgagccaaaaagtcatatacttggtcatgatactgctgattgccccggcatacagtatcaggtgcattggagtcagcaatagagacttcgtggagggcatgtcaggtgggacctgggttgatgttgtcttggaacatggaggctgcgttaccgtgatggcacaggacaagccaacagtcgacatagagttggtcacgacgacggttagtaacatggccgaggtaagatcctattgctacgaggcatcgatatcggacatggcttcggacagtcgttgcccaacacaaggtgaagcctaccttgacaagcaatcagacactcaatatgtctgcaaaagaacattagtggacagaggttggggaaacggttgtggactttttggcaaagggagcttggtgacatgtgccaagtttacgtgttctaagaagatgaccgggaagagcattcaaccggaaaatctggagtaccggataatgctatcagtgcatggctcccagcatagcgggatgattgttaatgacacaggatatgaaactgacgaaaatagagcgaaagtcgaggttacgcctaattcaccaagagcggaagcaaccttgggaggctttggaagcttaggacttgactgtgaaccaaggacaggccttgacttttcagatctgtattacctgaccatgaacaataagcattggttggtgcacaaagagtggtttcatgacatcccattgccttggcatgctggggcagacaccggaactccacactggaacaacaaagaggcattggtagaattcaaggatgcccacgccaagaggcaaaccgtcgtcgttctggggagccaggaaggagccgttcacacggctctcgctggagctctagaggctgagatggatggtgcaaagggaaagctgttctctggccatttgaaatgccgcctaaaaatggacaagcttagattgaagggcgtgtcatattccttgtgcactgcggcattcacattcaccaaggtcccagctgaaacactgcatggaacagtcacagtggaggtgcagtatgcagggacagatggaccctgcaagatcccagtccagatggcggtggacatgcagaccctgaccccagttggaaggctgataaccgccaaccccgtgattactgaaagcactgagaactcaaagatgatgttggagcttgacccaccatttggggattcttacattgtcataggagttggggacaagaaaatcacccaccactggcataggagtggtagcaccatcggaaaggcatttgaggccactgtgagaggcgccaagagaatggcagtcctgggggatacagcctgggacttcggatcagtcgggggtgtgttcaactcactgggtaagggcattcaccagatttttggagcagccttcaaatcactgtttggaggaatgtcctggttctcacagatcctcataggcacgctgctagtgtggttaggtttgaacacaaagaatggatctatctccctcacatgcttggccctggggggagtgatgatcttcctctccacggctgtttctgctgacgtggggtgctcagtggacttctcaaaaaaggaaacgagatgtggcacgggggtattcatctataatgatgttgaagcctggagggaccggtacaagtaccatcctgactccccccgcagattggcagcagcagtcaagcaggcctgggaagaggggatctgtgggatctcatccgtttcaagaatggaaaacatcatgtggaaatcagtagaaggggagctcaatgctatcctagaggagaatggagttcaactgacagttgttgtgggatctgtaaaaaaccccatgtggagaggtccacaaagattgccagtgcctgtgaatgagctgccccatggctggaaagcctgggggaaatcgtattttgttagggcggcaaagaccaacaacagttttgttgtcgacggtgacacactgaaggaatgtccgcttgagcacagagcatggaatagttttcttgtggaggatcacgggtttggagtcttccacaccagtgtctggcttaaggtcagagaagattactcattagaatgtgacccagccgtcataggaacagctgttaagggaagggaggccgcgcacagtgatctgggctattggattgaaagtgaaaagaatgacacatggaggctgaagagggcccacctgattgagatgaaaacatgtgaatggccaaagtctcacacattgtggacagatggagtagaagaaagtgatcttatcatacccaagtctttagctggtccactcagccaccacaacaccagagagggttacagaacccaagtgaaagggccatggcacagtgaagagcttgaaatccggtttgaggaatgtccaggcaccaaggtttacgtggaggagacatgcggaactagaggaccatctctgagatcaactactgcaagtggaagggtcattgaggaatggtgctgtagggaatgcacaatgcccccactatcgtttcgagcaaaagacggctgctggtatggaatggagataaggcccaggaaagaaccagagagcaacttagtgaggtcaatggtgacagcggggtcaaccgatcatatggaccacttctctcttggagtgcttgtgattctactcatggtgcaggaggggttgaagaagagaatgaccacaaagatcatcatgagcacatcaatggcagtgctggtagtcatgatcttgggaggattttcaatgagtgacctggccaagcttgtgatcctgatgggtgctactttcgcagaaatgaacactggaggagatgtagctcacttggcattggtagcggcatttaaagtcagaccagccttgctggtctccttcattttcagagccaattggacaccccgtgagagcatgctgctagccctggcttcgtgtcttctgcaaactgcgatctctgctcttgaaggtgacttgatggtcctcattaatggatttgctttggcctggttggcaattcgagcaatggccgtgccacgcactgacaacatcgctctaccaatcttggctgctctaacaccactagctcgaggcacactgctcgtggcatggagagcgggcctggctacttgtggagggatcatgctcctctccctgaaagggaaaggtagtgtgaagaagaacctgccatttgtcatggccctgggattgacagctgtgagggtagtagaccctattaatgtggtaggactactgttactcacaaggagtgggaagcggagctggccccctagtgaagttctcacagccgttggcctgatatgtgcactggccggagggtttgccaaggcagacattgagatggctggacccatggctgcagtaggcttgctaattgtcagctatgtggtctcgggaaagagtgtggacatgtacattgaaagagcaggtgacatcacatgggaaaaggacgcggaagtcactggaaacagtcctcggcttgacgtggcactggatgagagtggtgatttctccttggtagaggaagatggtccacccatgagagagatcatactcaaggtggtcctgatggccatctgtggcatgaacccaatagctataccttttgctgcaggagcgtggtatgtgtatgtgaagactgggaaaaggagtggcgccctctgggacgtgcctgctcccaaagaagtgaagaaaggagagaccacagatggagtgtacagagtgatgactcgcagactgctaggttcaacacaggttggagtgggagtcatgcaagagggagtcttccacaccatgtggcacgttacaaaaggagccgcactgaggagcggtgagggaagacttgatccatactggggggatgtcaagcaggacttggtgtcatactgtgggccttggaagttggatgcagcttgggatggactcagcgaggtacagcttttggccgtacctcccggagagagggccagaaacattcagaccctgcctggaatattcaagacaaaggacggggacatcggagcagttgctctggactaccctgcagggacctcaggatctccgatcctagacaaatgtggaagagtgataggactctatggcaatggggttgtgatcaagaatggaagctatgttagtgctataacccagggaaagagggaggaggagactccggttgaatgtttcgaaccctcgatgctgaagaagaagcagctaactgtcttggatctgcatccaggagccggaaaaaccaggagagttcttcctgaaatagtccgtgaagccataaaaaagagactccggacagtgatcttggcaccaactagggttgtcgctgctgagatggaggaggccttgagaggacttccggtgcgttacatgacaacagcagtcaacgtcacccattctgggacagaaatcgttgatttgatgtgccatgccactttcacttcacgcttactacaacccatcagagtccctaattacaatctctacatcatggatgaagcccacttcacagacccctcaagtatagctgcaagaggatacatatcaacaagggttgaaatgggcgaggcggctgccatttttatgactgccacaccaccaggaacccgtgatgcgtttcctgactctaactcaccaatcatggacacagaagtggaagtcccagagagagcctggagctcaggctttgattgggtgacagaccattctgggaaaacagtttggttcgttccaagcgtgagaaacggaaatgaaatcgcagcctgtctgacaaaggctggaaagcgggtcatacagctcagcaggaagacttttgagacagaatttcagaaaacaaaaaatcaagagtgggactttgtcataacaactgacatctcagagatgggcgccaacttcaaggctgaccgggtcatagactctaggagatgcctaaaaccagtcatacttgatggtgagagagtcatcttggctgggcccatgcctgtcacgcatgctagtgctgctcagaggagaggacgtataggcaggaaccctaacaaacctggagatgagtacatgtatggaggtgggtgtgcagagactgatgaaggccatgcacactggcttgaagcaagaatgcttcttgacaacatctacctccaggatggcctcatagcctcgctctatcggcctgaggccgataaggtagccgccattgagggagagtttaagctgaggacagagcaaaggaagaccttcgtggaactcatgaagagaggagaccttcccgtctggctagcctatcaggttgcatctgccggaataacttacacagacagaagatggtgctttgatggcacaaccaacaacaccataatggaagacagcgtaccagcagaggtgtggacaaagtatggagagaagagagtgctcaaaccgagatggatggatgctagggtctgttcagaccatgcggccctgaagtcgttcaaagaattcgccgctggaaaaagaggagcggctttgggagtaatggaggccctgggaacactgccaggacacatgacagagaggtttcaggaagccattgacaacctcgccgtgctcatgcgagcagagactggaagcaggccttataaggcagcggcagcccaactgccggagaccctagagaccattatgctcttaggtttgctgggaacagtttcactggggatcttcttcgtcttgatgcggaataagggcatcgggaagatgggctttggaatggtaacccttggggccagtgcatggctcatgtggctttcggaaattgaaccagccagaattgcatgtgtcctcattgttgtgtttttattactggtggtgctcatacccgagccagagaagcaaagatctccccaagataaccagatggcaattatcatcatggtggcagtgggccttctaggtttgataactgcaaacgaacttggatggctggaaagaacaaaaaatgacatagctcatctaatgggaaggagagaagaaggagcaaccatgggattctcaatggacattgatctgcggccagcctccgcctgggctatctatgccgcattgacaactctcatcaccccagctgtccaacatgcggtaaccacttcatacaacaactactccttaatggcgatggccacacaagctggagtgctgtttggcatgggcaaagggatgccattttatgcatgggaccttggagtcccgctgctaatgatgggttgctattcacaattaacacccctgactctgatagtagctatcattctgcttgtggcgcactacatgtacttgatcccaggcctacaagcggcagcagcgcgtgctgcccagaaaaggacagcagctggcatcatgaagaatcccgttgtggatggaatagtggtaactgacattgacacaatgacaatagacccccaggtggagaagaagatgggacaagtgttactcatagcagtagccatctccagtgctgtgctgctgcggaccgcctggggatggggggaggctggagctctgatcacagcagcgacctccaccttgtgggaaggctctccaaacaaatactggaactcctctacagccacctcactgtgcaacatcttcagaggaagctatctggcaggagcttcccttatctatacagtgacgagaaacgctggcctggttaagagacgtggaggtgggacgggagagactctgggagagaagtggaaagctcgtctgaatcagatgtcggccctggagttctactcttataaaaagtcaggtatcactgaagtgtgtagagaggaggctcgccgtgccctcaaggatggagtggccacaggaggacatgccgtatcccggggaagtgcaaagctcagatggttggtggagagaggatatctgcagccctatgggaaggttgttgacctcggatgtggcagagggggctggagctattatgccgccaccatccgcaaagtgcaggaggtgagaggatacacaaagggaggtcccggtcatgaagaacccatgctggtgcaaagctatgggtggaacatagttcgtctcaagagtggagtggacgtcttccacatggcggctgagccgtgtgacactctgctgtgtgacataggtgagtcatcatctagtcctgaagtggaagagacacgaacactcagagtgctctctatggtgggggactggcttgaaaaaagaccaggggccttctgtataaaggtgctgtgcccatacaccagcactatgatggaaaccatggagcgactgcaacgtaggcatgggggaggattagtcagagtgccattgtctcgcaactccacacatgagatgtactgggtctctggggcaaagagcaacatcataaaaagtgtgtccaccacaagtcagctcctcctgggacgcatggatggccccaggaggccagtgaaatatgaggaggatgtgaacctcggctcgggtacacgagctgtggcaagctgtgctgaggctcctaacatgaaaatcatcggcaggcgcattgagagaatccgcaatgaacatgcagaaacatggtttcttgatgaaaaccacccatacaggacatgggcctaccatgggagctacgaagcccccacgcaaggatcagcgtcttccctcgtgaacggggttgttagactcctgtcaaagccttgggacgtggtgactggagttacaggaatagccatgactgacaccacaccatacggccaacaaagagtcttcaaagaaaaagtggacaccagggtgccagatccccaagaaggcactcgccaggtaatgaacatagtctcttcctggctgtggaaggagctggggaaacgcaagcggccacgcgtctgcaccaaagaagagtttatcaacaaggtgcgcagcaatgcagcactgggagcaatatttgaagaggaaaaagaatggaagacggctgtggaagctgtgaatgatccaaggttttgggccctagtggatagggagagagaacaccacctgagaggagagtgtcacagctgtgtgtacaacatgatgggaaaaagagaaaagaagcaaggagagttcgggaaagcaaaaggtagccgcgccatctggtacatgtggttgggagccagattcttggagtttgaagcccttggattcttgaacgaggaccattggatgggaagagaaaactcaggaggtggagtcgaagggttaggattgcaaagacttggatacattctagaagaaatgaatcgggcaccaggaggaaagatgtacgcagatgacactgctggctgggacacccgcattagtaagtttgatctggagaatgaagctctgattaccaaccaaatggaggaagggcacagaactctggcgttggccgtgattaaatacacataccaaaacaaagtggtgaaggttctcagaccagctgaaggaggaaaaacagttatggacatcatttcaagacaagaccagagagggagtggacaagttgtcacttatgctctcaacacattcaccaacttggtggtgcagcttatccggaacatggaagctgaggaagtgttagagatgcaagacttatggttgttgaggaagccagagaaagtgaccagatggttgcagagcaatggatgggatagactcaaacgaatggcggtcagtggagatgactgcgttgtgaagccaatcgatgataggtttgcacatgccctcaggttcttgaatgacatgggaaaagttaggaaagacacacaggagtggaaaccctcgactggatggagcaattgggaagaagtcccgttctgctcccaccacttcaacaagctgtacctcaaggatgggagatccattgtggtcccttgccgccaccaagatgaactgattggccgagctcgcgtctcaccaggggcaggatggagcatccgggagactgcctgtcttgcaaaatcatatgcgcagatgtggcagctcctttatttccacagaagagaccttcgactgatggctaatgccatttgctcggctgtgccagttgactgggtaccaactgggagaaccacctggtcaatccatggaaagggagaatggatgaccactgaggacatgctcatggtgtggaatagagtgtggattgaggagaacgatcatatggaggacaagactcctgtaacaaaatggacagacattccctatctaggaaaaagggaggacttatggtgtggatcccttatagggcacagaccccgcaccacttgggctgaaaacatcaaagacacagtcaacatggtgcgcaggatcataggtgatgaagaaaagtacatggactatctatccacccaagtccgctacctgggtgaggaagggtccacacccggagtgttgtaagcaccaattttagtgttgtcaggcctgctagtcagccacagtttggggaaagctgtgcagcctgtaacccccccaggagaagctgggaaaccaagctcatagtcaggccgagaacgccatggcacggaagaagccatgctgcctgtgagcccctcagaggacactgagtcaaaaaaccccacgcgcttggaagcgcaggatgggaaaagaaggtggcgaccttccccacccttcaatctggggcctgaactggagactagctgtgaatctccagcagagggactagtggttagaggagaccccccggaaaacgcaaaacagcatattgacgctgggaaagaccagagactccatgagtttccaccacgctggccgccaggcacagatcgccgaacagcggcggccggtgtggggaaatccatggtttct

SEQ ID NO: 2 - DNA, 98 bp, synthetic construct
  source 1..98: mol_type = other DNA
atctgtgtgagtcagactgcgacagttcgagtctgaagcgagagctaacaacagtatcaacaggtttaatttggatttggaaacgagagtttctggtc

SEQ ID NO: 3 - DNA, 366 bp, synthetic construct
  source 1..366: mol_type = other DNA
atgaaaaacccaaagaagaaatccggaggattccggattgtcaatatgctaaaacgcggagtagcccgtgtaaaccccttgggaggtttgaagaggttgccagccggacttctgctgggtcatggacccatcagaatggttttggcgatactagcctttttgagatttacagcaatcaagccatcactgggccttatcaacagatggggttccgtggggaaaaaagaggctatggaaataataaagaagttcaagaaagatcttgctgccatgttgagaataatcaatgctaggaaagagaggaagagacgtggcgcagacaccagcatcggaatcattggcctcctgctgactacagccatggca

SEQ ID NO: 4 - DNA, 504 bp, synthetic construct
  source 1..504: mol_type = other DNA
gcagagatcactagacgcgggagtgcatactacatgtacttggataggagcgatgccgggaaggccatttcgtttgctaccacattgggagtgaacaagtgccacgtacagatcatggacctcgggcacatgtgtgacgccaccatgagttatgagtgccctatgctggatgagggagtggaaccagatgatgtcgattgctggtgcaacacgacatcaacttgggttgtgtacggaacctgtcatcacaaaaaaggtgaggcacggcgatctagaagagccgtgacgctcccttctcactctacaaggaagttgcaaacgcggtcgcagacctggttagaatcaagagaatacacgaagcacttgatcaaggttgaaaactggatattcaggaaccccgggtttgcgctagtggccgttgccattgcctggcttttgggaagctcgacgagccaaaaagtcatatacttggtcatgatactgctgattgccccggcatacagt

SEQ ID NO: 5 - DNA, 1512 bp, synthetic construct
  source 1..1512: mol_type = other DNA
atcaggtgcattggagtcagcaatagagacttcgtggagggcatgtcaggtgggacctgggttgatgttgtcttggaacatggaggctgcgttaccgtgatggcacaggacaagccaacagtcgacatagagttggtcacgacgacggttagtaacatggccgaggtaagatcctattgctacgaggcatcgatatcggacatggcttcggacagtcgttgcccaacacaaggtgaagcctaccttgacaagcaatcagacactcaatatgtctgcaaaagaacattagtggacagaggttggggaaacggttgtggactttttggcaaagggagcttggtgacatgtgccaagtttacgtgttctaagaagatgaccgggaagagcattcaaccggaaaatctggagtaccggataatgctatcagtgcatggctcccagcatagcgggatgattgttaatgacacaggatatgaaactgacgaaaatagagcgaaagtcgaggttacgcctaattcaccaagagcggaagcaaccttgggaggctttggaagcttaggacttgactgtgaaccaaggacaggccttgacttttcagatctgtattacctgaccatgaacaataagcattggttggtgcacaaagagtggtttcatgacatcccattgccttggcatgctggggcagacaccggaactccacactggaacaacaaagaggcattggtagaattcaaggatgcccacgccaagaggcaaaccgtcgtcgttctggggagccaggaaggagccgttcacacggctctcgctggagctctagaggctgagatggatggtgcaaagggaaagctgttctctggccatttgaaatgccgcctaaaaatggacaagcttagattgaagggcgtgtcatattccttgtgcactgcggcattcacattcaccaaggtcccagctgaaacactgcatggaacagtcacagtggaggtgcagtatgcagggacagatggaccctgcaagatcccagtccagatggcggtggacatgcagaccctgaccccagttggaaggctgataaccgccaaccccgtgattactgaaagcactgagaactcaaagatgatgttggagcttgacccaccatttggggattcttacattgtcataggagttggggacaagaaaatcacccaccactggcataggagtggtagcaccatcggaaaggcatttgaggccactgtgagaggcgccaagagaatggcagtcctgggggatacagcctgggacttcggatcagtcgggggtgtgttcaactcactgggtaagggcattcaccagatttttggagcagccttcaaatcactgtttggaggaatgtcctggttctcacagatcctcataggcacgctgctagtgtggttaggtttgaacacaaagaatggatctatctccctcacatgcttggccctggggggagtgatgatcttcctctccacggctgtttctgct

SEQ ID NO: 6 - DNA, 1056 bp, synthetic construct
  source 1..1056: mol_type = other DNA
gacgtggggtgctcagtggacttctcaaaaaaggaaacgagatgtggcacgggggtattcatctataatgatgttgaagcctggagggaccggtacaagtaccatcctgactccccccgcagattggcagcagcagtcaagcaggcctgggaagaggggatctgtgggatctcatccgtttcaagaatggaaaacatcatgtggaaatcagtagaaggggagctcaatgctatcctagaggagaatggagttcaactgacagttgttgtgggatctgtaaaaaaccccatgtggagaggtccacaaagattgccagtgcctgtgaatgagctgccccatggctggaaagcctgggggaaatcgtattttgttagggcggcaaagaccaacaacagttttgttgtcgacggtgacacactgaaggaatgtccgcttgagcacagagcatggaatagttttcttgtggaggatcacgggtttggagtcttccacaccagtgtctggcttaaggtcagagaagattactcattagaatgtgacccagccgtcataggaacagctgttaagggaagggaggccgcgcacagtgatctgggctattggattgaaagtgaaaagaatgacacatggaggctgaagagggcccacctgattgagatgaaaacatgtgaatggccaaagtctcacacattgtggacagatggagtagaagaaagtgatcttatcatacccaagtctttagctggtccactcagccaccacaacaccagagagggttacagaacccaagtgaaagggccatggcacagtgaagagcttgaaatccggtttgaggaatgtccaggcaccaaggtttacgtggaggagacatgcggaactagaggaccatctctgagatcaactactgcaagtggaagggtcattgaggaatggtgctgtagggaatgcacaatgcccccactatcgtttcgagcaaaagacggctgctggtatggaatggagataaggcccaggaaagaaccagagagcaacttagtgaggtcaatggtgacagcg

SEQ ID NO: 7 - DNA, 678 bp, synthetic construct
  source 1..678: mol_type = other DNA
gggtcaaccgatcatatggaccacttctctcttggagtgcttgtgattctactcatggtgcaggaggggttgaagaagagaatgaccacaaagatcatcatgagcacatcaatggcagtgctggtagtcatgatcttgggaggattttcaatgagtgacctggccaagcttgtgatcctgatgggtgctactttcgcagaaatgaacactggaggagatgtagctcacttggcattggtagcggcatttaaagtcagaccagccttgctggtctccttcattttcagagccaattggacaccccgtgagagcatgctgctagccctggcttcgtgtcttctgcaaactgcgatctctgctcttgaaggtgacttgatggtcctcattaatggatttgctttggcctggttggcaattcgagcaatggccgtgccacgcactgacaacatcgctctaccaatcttggctgctctaacaccactagctcgaggcacactgctcgtggcatggagagcgggcctggctacttgtggagggatcatgctcctctccctgaaagggaaaggtagtgtgaagaagaacctgccatttgtcatggccctgggattgacagctgtgagggtagtagaccctattaatgtggtaggactactgttactcacaaggagtgggaagcgg

SEQ ID NO: 8 - DNA, 390 bp, synthetic construct
  source 1..390: mol_type = other DNA
agctggccccctagtgaagttctcacagccgttggcctgatatgtgcactggccggagggtttgccaaggcagacattgagatggctggacccatggctgcagtaggcttgctaattgtcagctatgtggtctcgggaaagagtgtggacatgtacattgaaagagcaggtgacatcacatgggaaaaggacgcggaagtcactggaaacagtcctcggcttgacgtggcactggatgagagtggtgatttctccttggtagaggaagatggtccacccatgagagagatcatactcaaggtggtcctgatggccatctgtggcatgaacccaatagctataccttttgctgcaggagcgtggtatgtgtatgtgaagactgggaaaagg

SEQ ID NO: 9 - DNA, 1851 bp, synthetic construct
  source 1..1851: mol_type = other DNA
agtggcgccctctgggacgtgcctgctcccaaagaagtgaagaaaggagagaccacagatggagtgtacagagtgatgactcgcagactgctaggttcaacacaggttggagtgggagtcatgcaagagggagtcttccacaccatgtggcacgttacaaaaggagccgcactgaggagcggtgagggaagacttgatccatactggggggatgtcaagcaggacttggtgtcatactgtgggccttggaagttggatgcagcttgggatggactcagcgaggtacagcttttggccgtacctcccggagagagggccagaaacattcagaccctgcctggaatattcaagacaaaggacggggacatcggagcagttgctctggactaccctgcagggacctcaggatctccgatcctagacaaatgtggaagagtgataggactctatggcaatggggttgtgatcaagaatggaagctatgttagtgctataacccagggaaagagggaggaggagactccggttgaatgtttcgaaccctcgatgctgaagaagaagcagctaactgtcttggatctgcatccaggagccggaaaaaccaggagagttcttcctgaaatagtccgtgaagccataaaaaagagactccggacagtgatcttggcaccaactagggttgtcgctgctgagatggaggaggccttgagaggacttccggtgcgttacatgacaacagcagtcaacgtcacccattctgggacagaaatcgttgatttgatgtgccatgccactttcacttcacgcttactacaacccatcagagtccctaattacaatctctacatcatggatgaagcccacttcacagacccctcaagtatagctgcaagaggatacatatcaacaagggttgaaatgggcgaggcggctgccatttttatgactgccacaccaccaggaacccgtgatgcgtttcctgactctaactcaccaatcatggacacagaagtggaagtcccagagagagcctggagctcaggctttgattgggtgacagaccattctgggaaaacagtttggttcgttccaagcgtgagaaacggaaatgaaatcgcagcctgtctgacaaaggctggaaagcgggtcatacagctcagcaggaagacttttgagacagaatttcagaaaacaaaaaatcaagagtgggactttgtcataacaactgacatctcagagatgggcgccaacttcaaggctgaccgggtcatagactctaggagatgcctaaaaccagtcatacttgatggtgagagagtcatcttggctgggcccatgcctgtcacgcatgctagtgctgctcagaggagaggacgtataggcaggaaccctaacaaacctggagatgagtacatgtatggaggtgggtgtgcagagactgatgaaggccatgcacactggcttgaagcaagaatgcttcttgacaacatctacctccaggatggcctcatagcctcgctctatcggcctgaggccgataaggtagccgccattgagggagagtttaagctgaggacagagcaaaggaagaccttcgtggaactcatgaagagaggagaccttcccgtctggctagcctatcaggttgcatctgccggaataacttacacagacagaagatggtgctttgatggcacaaccaacaacaccataatggaagacagcgtaccagcagaggtgtggacaaagtatggagagaagagagtgctcaaaccgagatggatggatgctagggtctgttcagaccatgcggccctgaagtcgttcaaagaattcgccgctggaaaaaga

SEQ ID NO: 10 - DNA, 381 bp, synthetic construct
  source 1..381: mol_type = other DNA
ggagcggctttgggagtaatggaggccctgggaacactgccaggacacatgacagagaggtttcaggaagccattgacaacctcgccgtgctcatgcgagcagagactggaagcaggccttataaggcagcggcagcccaactgccggagaccctagagaccattatgctcttaggtttgctgggaacagtttcactggggatcttcttcgtcttgatgcggaataagggcatcgggaagatgggctttggaatggtaacccttggggccagtgcatggctcatgtggctttcggaaattgaaccagccagaattgcatgtgtcctcattgttgtgtttttattactggtggtgctcatacccgagccagagaagcaaaga

SEQ ID NO: 11 - DNA, 822 bp, synthetic construct
  source 1..822: mol_type = other DNA
tctccccaagataaccagatggcaattatcatcatggtggcagtgggccttctaggtttgataactgcaaacgaacttggatggctggaaagaacaaaaaatgacatagctcatctaatgggaaggagagaagaaggagcaaccatgggattctcaatggacattgatctgcggccagcctccgcctgggctatctatgccgcattgacaactctcatcaccccagctgtccaacatgcggtaaccacttcatacaacaactactccttaatggcgatggccacacaagctggagtgctgtttggcatgggcaaagggatgccattttatgcatgggaccttggagtcccgctgctaatgatgggttgctattcacaattaacacccctgactctgatagtagctatcattctgcttgtggcgcactacatgtacttgatcccaggcctacaagcggcagcagcgcgtgctgcccagaaaaggacagcagctggcatcatgaagaatcccgttgtggatggaatagtggtaactgacattgacacaatgacaatagacccccaggtggagaagaagatgggacaagtgttactcatagcagtagccatctccagtgctgtgctgctgcggaccgcctggggatggggggaggctggagctctgatcacagcagcgacctccaccttgtgggaaggctctccaaacaaatactggaactcctctacagccacctcactgtgcaacatcttcagaggaagctatctggcaggagcttcccttatctatacagtgacgagaaacgctggcctggttaagagacgt

SEQ ID NO: 12 - DNA, 2709 bp, synthetic construct
  source 1..2709: mol_type = other DNA
ggaggtgggacgggagagactctgggagagaagtggaaagctcgtctgaatcagatgtcggccctggagttctactcttataaaaagtcaggtatcactgaagtgtgtagagaggaggctcgccgtgccctcaaggatggagtggccacaggaggacatgccgtatcccggggaagtgcaaagctcagatggttggtggagagaggatatctgcagccctatgggaaggttgttgacctcggatgtggcagagggggctggagctattatgccgccaccatccgcaaagtgcaggaggtgagaggatacacaaagggaggtcccggtcatgaagaacccatgctggtgcaaagctatgggtggaacatagttcgtctcaagagtggagtggacgtcttccacatggcggctgagccgtgtgacactctgctgtgtgacataggtgagtcatcatctagtcctgaagtggaagagacacgaacactcagagtgctctctatggtgggggactggcttgaaaaaagaccaggggccttctgtataaaggtgctgtgcccatacaccagcactatgatggaaaccatggagcgactgcaacgtaggcatgggggaggattagtcagagtgccattgtctcgcaactccacacatgagatgtactgggtctctggggcaaagagcaacatcataaaaagtgtgtccaccacaagtcagctcctcctgggacgcatggatggccccaggaggccagtgaaatatgaggaggatgtgaacctcggctcgggtacacgagctgtggcaagctgtgctgaggctcctaacatgaaaatcatcggcaggcgcattgagagaatccgcaatgaacatgcagaaacatggtttcttgatgaaaaccacccatacaggacatgggcctaccatgggagctacgaagcccccacgcaaggatcagcgtcttccctcgtgaacggggttgttagactcctgtcaaagccttgggacgtggtgactggagttacaggaatagccatgactgacaccacaccatacggccaacaaagagtcttcaaagaaaaagtggacaccagggtgccagatccccaagaaggcactcgccaggtaatgaacatagtctcttcctggctgtggaaggagctggggaaacgcaagcggccacgcgtctgcaccaaagaagagtttatcaacaaggtgcgcagcaatgcagcactgggagcaatatttgaagaggaaaaagaatggaagacggctgtggaagctgtgaatgatccaaggttttgggccctagtggatagggagagagaacaccacctgagaggagagtgtcacagctgtgtgtacaacatgatgggaaaaagagaaaagaagcaaggagagttcgggaaagcaaaaggtagccgcgccatctggtacatgtggttgggagccagattcttggagtttgaagcccttggattcttgaacgaggaccattggatgggaagagaaaactcaggaggtggagtcgaagggttaggattgcaaagacttggatacattctagaagaaatgaatcgggcaccaggaggaaagatgtacgcagatgacactgctggctgggacacccgcattagtaagtttgatctggagaatgaagctctgattaccaaccaaatggaggaagggcacagaactctggcgttggccgtgattaaatacacataccaaaacaaagtggtgaaggttctcagaccagctgaaggaggaaaaacagttatggacatcatttcaagacaagaccagagagggagtggacaagttgtcacttatgctctcaacacattcaccaacttggtggtgcagcttatccggaacatggaagctgaggaagtgttagagatgcaagacttatggttgttgaggaagccagagaaagtgaccagatggttgcagagcaatggatgggatagactcaaacgaatggcggtcagtggagatgactgcgttgtgaagccaatcgatgataggtttgcacatgccctcaggttcttgaatgacatgggaaaagttaggaaagacacacaggagtggaaaccctcgactggatggagcaattgggaagaagtcccgttctgctcccaccacttcaacaagctgtacctcaaggatgggagatccattgtggtcccttgccgccaccaagatgaactgattggccgagctcgcgtctcaccaggggcaggatggagcatccgggagactgcctgtcttgcaaaatcatatgcgcagatgtggcagctcctttatttccacagaagagaccttcgactgatggctaatgccatttgctcggctgtgccagttgactgggtaccaactgggagaaccacctggtcaatccatggaaagggagaatggatgaccactgaggacatgctcatggtgtggaatagagtgtggattgaggagaacgatcatatggaggacaagactcctgtaacaaaatggacagacattccctatctaggaaaaagggaggacttatggtgtggatcccttatagggcacagaccccgcaccacttgggctgaaaacatcaaagacacagtcaacatggtgcgcaggatcataggtgatgaagaaaagtacatggactatctatccacccaagtccgctacctgggtgaggaagggtccacacccggagtgttg

SEQ ID NO: 13 - DNA, 432 bp, synthetic construct
  source 1..432: mol_type = other DNA
taagcaccaattttagtgttgtcaggcctgctagtcagccacagtttggggaaagctgtgcagcctgtaacccccccaggagaagctgggaaaccaagctcatagtcaggccgagaacgccatggcacggaagaagccatgctgcctgtgagcccctcagaggacactgagtcaaaaaaccccacgcgcttggaagcgcaggatgggaaaagaaggtggcgaccttccccacccttcaatctggggcctgaactggagactagctgtgaatctccagcagagggactagtggttagaggagaccccccggaaaacgcaaaacagcatattgacgctgggaaagaccagagactccatgagtttccaccacgctggccgccaggcacagatcgccgaacagcggcggccggtgtggggaaatccatggtttct

SEQ ID NO: 14 - protein, 8 aa, synthetic construct
  VAR_SEQ 2: note = X is V or I
  VAR_SEQ 4: note = X is any amino acid
DXEXNPGP

SEQ ID NO: 15 - DNA, 4361 bp, synthetic construct
  source 1..4361: mol_type = other DNA
ttctcatgtttgacagcttatcatcgataagctttaatgcggtagtttatcacagttaaattgctaacgcagtcaggcaccgtgtatgaaatctaacaatgcgctcatcgtcatcctcggcaccgtcaccctggatgctgtaggcataggcttggttatgccggtactgccgggcctcttgcgggatatcgtccattccgacagcatcgccagtcactatggcgtgctgctagcgctatatgcgttgatgcaatttctatgcgcacccgttctcggagcactgtccgaccgctttggccgccgcccagtcctgctcgcttcgctacttggagccactatcgactacgcgatcatggcgaccacacccgtcctgtggatcctctacgccggacgcatcgtggccggcatcaccggcgccacaggtgcggttgctggcgcctatatcgccgacatcaccgatggggaagatcgggctcgccacttcgggctcatgagcgcttgtttcggcgtgggtatggtggcaggccccgtggccgggggactgttgggcgccatctccttgcatgcaccattccttgcggcggcggtgctcaacggcctcaacctactactgggctgcttcctaatgcaggagtcgcataagggagagcgtcgaccgatgcccttgagagccttcaacccagtcagctccttccggtgggcgcggggcatgactatcgtcgccgcacttatgactgtcttctttatcatgcaactcgtaggacaggtgccggcagcgctctgggtcattttcggcgaggaccgctttcgctggagcgcgacgatgatcggcctgtcgcttgcggtattcggaatcttgcacgccctcgctcaagccttcgtcactggtcccgccaccaaacgtttcggcgagaagcaggccattatcgccggcatggcggccgacgcgctgggctacgtcttgctggcgttcgcgacgcgaggctggatggccttccccattatgattcttctcgcttccggcggcatcgggatgcccgcgttgcaggccatgctgtccaggcaggtagatgacgaccatcagggacagcttcaaggatcgctcgcggctcttaccagcctaacttcgatcattggaccgctgatcgtcacggcgatttatgccgcctcggcgagcacatggaacgggttggcatggattgtaggcgccgccctataccttgtctgcctccccgcgttgcgtcgcggtgcatggagccgggccacctcgacctgaatggaagccggcggcacctcgctaacggattcaccactccaagaattggagccaatcaattcttgcggagaactgtgaatgcgcaaaccaacccttggcagaacatatccatcgcgtccgccatctccagcagccgcacgcggcgcatctcgggcagcgttgggtcctggccacgggtgcgcatgatcgtgctcctgtcgttgaggacccggctaggctggcggggttgccttactggttagcagaatgaatcaccgatacgcgagcgaacgtgaagcgactgctgctgcaaaacgtctgcgacctgagcaacaacatgaatggtcttcggtttccgtgtttcgtaaagtctggaaacgcggaagtcagcgccctgcaccattatgttccggatctgcatcgcaggatgctgctggctaccctgtggaacacctacatctgtattaacgaagcgctggcattgaccctgagtgatttttctctggtcccgccgcatccataccgccagttgtttaccctcacaacgttccagtaaccgggcatgttcatcatcagtaacccgtatcgtgagcatcctctctcgtttcatcggtatcattacccccatgaacagaaatcccccttacacggaggcatcagtgaccaaacaggaaaaaaccgcccttaacatggcccgctttatcagaagccagacattaacgcttctggagaaactcaacgagctggacgcggatgaacaggcagacatctgtgaatcgcttcacgaccacgctgatgagctttaccgcagctgcctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggcgcagccatgacccagtcacgtagcgatagcggagtgtatactggcttaactatgcggcatcagagcagattgtactgagagtgcaccatatgcggtgtgaaataccgcacagatgcgtaaggagaaaataccgcatcaggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctgcaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaacacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtcttcaagaa

SEQ ID NO: 16 - DNA, 11970 bp, synthetic construct
  source 1..11970: mol_type = other DNA
gacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctcgtttagtgaaccgtagttgttgatctgtgtgagtcagactgcgacagttcgagtctgaagcgagagctaacaacagtatcaacaggtttaatttggatttggaaacgagagtttctggtcatgaaaaacccaaagaagaaatccggaggattccggattgtcaatatgctaaaacgcggagtagcccgtgtaaaccccttgggaggtttgaagaggttgccagccggacttctgctgggtcatggacccatcagaatggttttggcgatactagcctttttgagatttacagcaatcaagccatcactgggccttatcaacagatggggttccgtggggaaaaaagaggctatggaaataataaagaagttcaagaaagatcttgctgccatgttgagaataatcaatgctaggaaagagaggaagagacgtggcgcagacaccagcatcggaatcattggcctcctgctgactacagccatggcagcagagatcactagacgcgggagtgcatactacatgtacttggataggagcgatgccgggaaggccatttcgtttgctaccacattgggagtgaacaagtgccacgtacagatcatggacctcgggcacatgtgtgacgccaccatgagttatgagtgccctatgctggatgagggagtggaaccagatgatgtcgattgctggtgcaacacgacatcaacttgggttgtgtacggaacctgtcatcacaaaaaaggtgaggcacggcgatctagaagagccgtgacgctcccttctcactctacaaggaagttgcaaacgcggtcgcagacctggttagaatcaagagaatacacgaagcacttgatcaaggttgaaaactggatattcaggaaccccgggtttgcgctagtggccgttgccattgcctggcttttgggaagctcgacgagccaaaaagtcatatacttggtcatgatactgctgattgccccggcatacagtatcaggtgcattggagtcagcaatagagacttcgtggagggcatgtcaggtgggacctgggttgatgttgtcttggaacatggaggctgcgttaccgtgatggcacaggacaagccaacagtcgacatagagttggtcacgacgacggttagtaacatggccgaggtaagatcctattgctacgaggcatcgatatcggacatggcttcggacagtcgttgcccaacacaaggtgaagcctaccttgacaagcaatcagacactcaatatgtctgcaaaagaacattagtggacagaggttggggaaacggttgtggactttttggcaaagggagcttggtgacatgtgccaagtttacgtgttctaagaagatgaccgggaagagcattcaaccggaaaatctggagtaccggataatgctatcagtgcatggctcccagcatagcgggatgattgttaatgacacaggatatgaaactgacgaaaatagagcgaaagtcgaggttacgcctaattcaccaagagcggaagcaaccttgggaggctttggaagcttaggacttgactgtgaaccaaggacaggccttgacttttcagatctgtattacctgaccatgaacaataagcattggttggtgcacaaagagtggtttcatgacatcccattgccttggcatgctggggcagacaccggaactccacactggaacaacaaagaggcattggtagaattcaaggatgcccacgccaagaggcaaaccgtcgtcgttctggggagccaggaaggagccgttcacacggctctcgctggagctctagaggctgagatggatggtgcaaagggaaagctgttctctggccatttgaaatgccgcctaaaaatggacaagcttagattgaagggcgtgtcatattccttgtgcactgcggcattcacattcaccaaggtcccagctgaaacactgcatggaacagtcacagtggaggtgcagtatgcagggacagatggaccctgcaagatcccagtccagatggcggtggacatgcagaccctgaccccagttggaaggctgataaccgccaaccccgtgattactgaaagcactgagaactcaaagatgatgttggagcttgacccaccatttggggattcttacattgtcataggagttggggacaagaaaatcacccaccactggcataggagtggtagcaccatcggaaaggcatttgaggccactgtgagaggcgccaagagaatggcagtcctgggggatacagcctgggacttcggatcagtcgggggtgtgttcaactcactgggtaagggcattcaccagatttttggagcagccttcaaatcactgtttggaggaatgtcctggttctcacagatcctcataggcacgctgctagtgtggttaggtttgaacacaaagaatggatctatctccctcacatgcttggccctggggggagtgatgatcttcctctccacggctgtttctgctgacgtggggtgctcagtggacttctcaaaaaaggaaacgagatgtggcacgggggtattcatctataatgatgttgaagcctggagggaccggtacaagtaccatcctgactccccccgcagattggcagcagcagtcaagcaggcctgggaagaggggatctgtgggatctcatccgtttcaagaatggaaaacatcatgtggaaatcagtagaaggggagctcaatgctatcctagaggagaatggagttcaactgacagttgttgtgggatctgtaaaaaaccccatgtggagaggtccacaaagattgccagtgcctgtgaatgagctgccccatggctggaaagcctgggggaaatcgtattttgttagggcggcaaagaccaacaacagttttgttgtcgacggtgacacactgaaggaatgtccgcttgagcacagagcatggaatagttttcttgtggaggatcacgggtttggagtcttccacaccagtgtctggcttaaggtcagagaagattactcattagaatgtgacccagccgtcataggaacagctgttaagggaagggaggccgcgcacagtgatctgggctattggattgaaagtgaaaagaatgacacatggaggctgaaggtaagtatcaaggttacaagacaggtttaaggagaccaatagaaactgggcttgtcgagacagagaagactcttgcgtttctgataggcacctattggtcttactgacatccactttgcctttctctccacagagggcccacctgattgagatgaaaacatgtgaatggccaaagtctcacacattgtggacagatggagtagaagaaagtgatcttatcatacccaagtctttagctggtccactcagccaccacaacaccagagagggttacagaacccaagtgaaagggccatggcacagtgaagagcttgaaatccggtttgaggaatgtccaggcaccaaggtttacgtggaggagacatgcggaactagaggaccatctctgagatcaactactgcaagtggaagggtcattgaggaatggtgctgtagggaatgcacaatgcccccactatcgtttcgagcaaaagacggctgctggtatggaatggagataaggcccaggaaagaaccagagagcaacttagtgaggtcaatggtgacagcggggtcaaccgatcatatggaccacttctctcttggagtgcttgtgattctactcatggtgcaggaggggttgaagaagagaatgaccacaaagatcatcatgagcacatcaatggcagtgctggtagtcatgatcttgggaggattttcaatgagtgacctggccaagcttgtgatcctgatgggtgctactttcgcagaaatgaacactggaggagatgtagctcacttggcattggtagcggcatttaaagtcagaccagccttgctggtctccttcattttcagagccaattggacaccccgtgagagcatgctgctagccctggcttcgtgtcttctgcaaactgcgatctctgctcttgaaggtgacttgatggtcctcattaatggatttgctttggcctggttggcaattcgagcaatggccgtgccacgcactgacaacatcgctctaccaatcttggctgctctaacaccactagctcgaggcacactgctcgtggcatggagagcgggcctggctacttgtggagggatcatgctcctctccctgaaagggaaaggtagtgtgaagaagaacctgccatttgtcatggccctgggattgacagctgtgagggtagtagaccctattaatgtggtaggactactgttactcacaaggagtgggaagcggagctggccccctagtgaagttctcacagccgttggcctgatatgtgcactggccggagggtttgccaaggcagacattgagatggctggacccatggctgcagtaggcttgctaattgtcagctatgtggtctcgggaaagagtgtggacatgtacattgaaagagcaggtgacatcacatgggaaaaggacgcggaagtcactggaaacagtcctcggcttgacgtggcactggatgagagtggtgatttctccttggtagaggaagatggtccacccatgagagagatcatactcaaggtggtcctgatggccatctgtggcatgaacccaatagctataccttttgctgcaggagcgtggtatgtgtatgtgaagactgggaaaaggagtggcgccctctgggacgtgcctgctcccaaagaagtgaagaaaggagagaccacagatggagtgtacagagtgatgactcgcagactgctaggttcaacacaggttggagtgggagtcatgcaagagggagtcttccacaccatgtggcacgttacaaaaggagccgcactgaggagcggtgagggaagacttgatccatactggggggatgtcaagcaggacttggtgtcatactgtgggccttggaagttggatgcagcttgggatggactcagcgaggtacagcttttggccgtacctcccggagagagggccagaaacattcagaccctgcctggaatattcaagacaaaggacggggacatcggagcagttgctctggactaccctgcagggacctcaggatctccgatcctagacaaatgtggaagagtgataggactctatggcaatggggttgtgatcaagaatggaagctatgttagtgctataacccagggaaagagggaggaggagactccggttgaatgtttcgaaccctcgatgctgaagaagaagcagctaactgtcttggatctgcatccaggagccggaaaaaccaggagagttcttcctgaaatagtccgtgaagccataaaaaagagactccggacagtgatcttggcaccaactagggttgtcgctgctgagatggaggaggccttgagaggacttccggtgcgttacatgacaacagcagtcaacgtcacccattctgggacagaaatcgttgatttgatgtgccatgccactttcacttcacgcttactacaacccatcagagtccctaattacaatctctacatcatggatgaagcccacttcacagacccctcaagtatagctgcaagaggatacatatcaacaagggttgaaatgggcgaggcggctgccatttttatgactgccacaccaccaggaacccgtgatgcgtttcctgactctaactcaccaatcatggacacagaagtggaagtcccagagagagcctggagctcaggctttgattgggtgacagaccattctgggaaaacagtttggttcgttccaagcgtgagaaacggaaatgaaatcgcagcctgtctgacaaaggctggaaagcgggtcatacagctcagcaggaagacttttgagacagaatttcagaaaacaaaaaatcaagagtgggactttgtcataacaactgacatctcagagatgggcgccaacttcaaggctgaccgggtcatagactctaggagatgcctaaaaccagtcatacttgatggtgagagagtcatcttggctgggcccatgcctgtcacgcatgctagtgctgctcagaggagaggacgtataggcaggaaccctaacaaacctggagatgagtacatgtatggaggtgggtgtgcagagactgatgaaggccatgcacactggcttgaagcaagaatgcttcttgacaacatctacctccaggatggcctcatagcctcgctctatcggcctgaggccgataaggtagccgccattgagggagagtttaagctgaggacagagcaaaggaagaccttcgtggaactcatgaagagaggagaccttcccgtctggctagcctatcaggttgcatctgccggaataacttacacagacagaagatggtgctttgatggcacaaccaacaacaccataatggaagacagcgtaccagcagaggtgtggacaaagtatggagagaagagagtgctcaaaccgagatggatggatgctagggtctgttcagaccatgcggccctgaagtcgttcaaagaattcgccgctggaaaaagaggagcggctttgggagtaatggaggccctgggaacactgccaggacacatgacagagaggtttcaggaagccattgacaacctcgccgtgctcatgcgagcagagactggaagcaggccttataaggcagcggcagcccaactgccggagaccctagagaccattatgctcttaggtttgctgggaacagtttcactggggatcttcttcgtcttgatgcggaataagggcatcgggaagatgggctttggaatggtaacccttggggccagtgcatggctcatgtggctttcggaaattgaaccagccagaattgcatgtgtcctcattgttgtgtttttattactggtggtgctcatacccgagccagagaagcaaagatctccccaagataaccagatggcaattatcatcatggtggcagtgggccttctaggtttgataactgcaaacgaacttggatggctggaaagaacaaaaaatgacatagctcatctaatgggaaggagagaagaaggagcaaccatgggattctcaatggacattgatctgcggccagcctccgcctgggctatctatgccgcattgacaactctcatcaccccagctgtccaacatgcggtaaccacttcatacaacaactactccttaatggcgatggccacacaagctggagtgctgtttggcatgggcaaagggatgccattttatgcatgggaccttggagtcccgctgctaatgatgggttgctattcacaattaacacccctgactctgatagtagctatcattctgcttgtggcgcactacatgtacttgatcccaggcctacaagcggcagcagcgcgtgctgcccagaaaaggacagcagctggcatcatgaagaatcccgttgtggatggaatagtggtaactgacattgacacaatgacaatagacccccaggtggagaagaagatgggacaagtgttactcatagcagtagccatctccagtgctgtgctgctgcggaccgcctggggatggggggaggctggagctctgatcacagcagcgacctccaccttgtgggaaggctctccaaacaaatactggaactcctctacagccacctcactgtgcaacatcttcagaggaagctatctggcaggagcttcccttatctatacagtgacgagaaacgctggcctggttaagagacgtggaggtgggacgggagagactctgggagagaagtggaaagctcgtctgaatcagatgtcggccctggagttctactcttataaaaagtcaggtatcactgaagtgtgtagagaggaggctcgccgtgccctcaaggatggagtggccacaggaggacatgccgtatcccggggaagtgcaaagctcagatggttggtggagagaggatatctgcagccctatgggaaggttgttgacctcggatgtggcagagggggctggagctattatgccgccaccatccgcaaagtgcaggaggtgagaggatacacaaagggaggtcccggtcatgaagaacccatgctggtgcaaagctatgggtggaacatagttcgtctcaagagtggagtggacgtcttccacatggcggctgagccgtgtgacactctgctgtgtgacataggtgagtcatcatctagtcctgaagtggaagagacacgaacactcagagtgctctctatggtgggggactggcttgaaaaaagaccaggggccttctgtataaaggtgctgtgcccatacaccagcactatgatggaaaccatggagcgactgcaacgtaggcatgggggaggattagtcagagtgccattgtctcgcaactccacacatgagatgtactgggtctctggggcaaagagcaacatcataaaaagtgtgtccaccacaagtcagctcctcctgggacgcatggatggccccaggaggccagtgaaatatgaggaggatgtgaacctcggctcgggtacacgagctgtggcaagctgtgctgaggctcctaacatgaaaatcatcggcaggcgcattgagagaatccgcaatgaacatgcagaaacatggtttcttgatgaaaaccacccatacaggacatgggcctaccatgggagctacgaagcccccacgcaaggatcagcgtcttccctcgtgaacggggttgttagactcctgtcaaagccttgggacgtggtgactggagttacaggaatagccatgactgacaccacaccatacggccaacaaagagtcttcaaagaaaaagtggacaccagggtgccagatccccaagaaggcactcgccaggtaatgaacatagtctcttcctggctgtggaaggagctggggaaacgcaagcggccacgcgtctgcaccaaagaagagtttatcaacaaggtgcgcagcaatgcagcactgggagcaatatttgaagaggaaaaagaatggaagacggctgtggaagctgtgaatgatccaaggttttgggccctagtggatagggagagagaacaccacctgagaggagagtgtcacagctgtgtgtacaacatgatgggaaaaagagaaaagaagcaaggagagttcgggaaagcaaaaggtagccgcgccatctggtacatgtggttgggagccagattcttggagtttgaagcccttggattcttgaacgaggaccattggatgggaagagaaaactcaggaggtggagtcgaagggttaggattgcaaagacttggatacattctagaagaaatgaatcgggcaccaggaggaaagatgtacgcagatgacactgctggctgggacacccgcattagtaagtttgatctggagaatgaagctctgattaccaaccaaatggaggaagggcacagaactctggcgttggccgtgattaaatacacataccaaaacaaagtggtgaaggttctcagaccagctgaaggaggaaaaacagttatggacatcatttcaagacaagaccagagagggagtggacaagttgtcacttatgctctcaacacattcaccaacttggtggtgcagcttatccggaacatggaagctgaggaagtgttagagatgcaagacttatggttgttgaggaagccagagaaagtgaccagatggttgcagagcaatggatgggatagactcaaacgaatggcggtcagtggagatgactgcgttgtgaagccaatcgatgataggtttgcacatgccctcaggttcttgaatgacatgggaaaagttaggaaagacacacaggagtggaaaccctcgactggatggagcaattgggaagaagtcccgttctgctcccaccacttcaacaagctgtacctcaaggatgggagatccattgtggtcccttgccgccaccaagatgaactgattggccgagctcgcgtctcaccaggggcaggatggagcatccgggagactgcctgtcttgcaaaatcatatgcgcagatgtggcagctcctttatttccacagaagagaccttcgactgatggctaatgccatttgctcggctgtgccagttgactgggtaccaactgggagaaccacctggtcaatccatggaaagggagaatggatgaccactgaggacatgctcatggtgtggaatagagtgtggattgaggagaacgatcatatggaggacaagactcctgtaacaaaatggacagacattccctatctaggaaaaagggaggacttatggtgtggatcccttatagggcacagaccccgcaccacttgggctgaaaacatcaaagacacagtcaacatggtgcgcaggatcataggtgatgaagaaaagtacatggactatctatccacccaagtccgctacctgggtgaggaagggtccacacccggagtgttgtaagcaccaattttagtgttgtcaggcctgctagtcagccacagtttggggaaagctgtgcagcctgtaacccccccaggagaagctgggaaaccaagctcatagtcaggccgagaacgccatggcacggaagaagccatgctgcctgtgagcccctcagaggacactgagtcaaaaaaccccacgcgcttggaagcgcaggatgggaaaagaaggtggcgaccttccccacccttcaatctggggcctgaactggagactagctgtgaatctccagcagagggactagtggttagaggagaccccccggaaaacgcaaaacagcatattgacgctgggaaagaccagagactccatgagtttccaccacgctggccgccaggcacagatcgccgaacagcggcggccggtgtggggaaatccatggtttctggccggcatggtcccagcctcctcgctggcgccggctgggcaacattccgaggggaccgtcccctcggtaatggcgaatgggacccgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggaatcgatagtactaacatacgctctccatcaaaacaaaacgaaacaaaacaaactagcaaaataggctgtccccagtgcaagtgcaggtgccagaacatttctct

SEQ ID NO: 17 - DNA, 599 bp, synthetic construct
  source 1..599: mol_type = other DNA
gacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctcgtttagtgaaccgt

SEQ ID NO: 18 - DNA, 261 bp, synthetic construct
  source 1..261: mol_type = other DNA
gggtcggcatggcatctccacctcctcgcggtccgacctgggcatccgaaggaggacgtcgtccactcggatggctaagggagagccacttttctctcgattctctatcggaatctagggagctcggatccagacatgataagatacattgatgagtttggacaaaccacaactagaatgcagtgaaaaaaatgctttatttgtgaaatttgtgatgctattgctttatttgtaaccattataagctgcaataaacaagtt

SEQ ID NO: 19 - DNA, 326 bp, synthetic construct
  source 1..326: mol_type = other DNA
ggccggcatggtcccagcctcctcgctggcgccggctgggcaacattccgaggggaccgtcccctcggtaatggcgaatgggacccgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatgg

SEQ ID NO: 20 - DNA, 341 bp, synthetic construct
  source 1..341: mol_type = other DNA
ggccggcatggtcccagcctcctcgctggcgccggctgggcaacattccgaggggaccgtcccctcggtaatggcgaatgggacccgctgatcagcctcgagatccagacatgataagatacattgatgagtttggacaaaccacaactagaatgcagtgaaaaaaatgctttatttgtgaaatttgtgatgctattgctttatttgtaaccattataagctgcaataaacaagttaatcgatagtactaacatacgctctccatcaaaacaaaacgaaacaaaacaaactagcaaaataggctgtccccagtgcaagtgcaggtgccagaacatttctct

SEQ ID NO: 21 - DNA, 431 bp, synthetic construct
  source 1..431: mol_type = other DNA
ggccggcatggtcccagcctcctcgctggcgccggctgggcaacattccgaggggaccgtcccctcggtaatggcgaatgggacccgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggaatcgatagtactaacatacgctctccatcaaaacaaaacgaaacaaaacaaactagcaaaataggctgtccccagtgcaagtgcaggtgccagaacatttctct

SEQ ID NO: 22 - DNA, 133 bp, synthetic construct
  source 1..133: mol_type = other DNA
gtaagtatcaaggttacaagacaggtttaaggagaccaatagaaactgggcttgtcgagacagagaagactcttgcgtttctgataggcacctattggtcttactgacatccactttgcctttctctccacag